SEQUENCE LISTING (9 sequences)

SEQ ID NO: 1 - DNA, 6444 bp, Homo sapiens
atgaggagcttcaaaagagtcaactttgggactctgctaagcagccagaaggaggctgaagagttgctgcccgacttgaaggtgggggtgctgcctggctcgggatacacctggctttccaaactgagctgttttgtgtttgccttttgaagagatggataagagttcctgtccaaccctccagctggttttcccagcagccgatctgatgctgagaggagacaagcttgtgatgccatcctgagggcttgcaaccagcagctgactgctaagctagcttgccctaggcatctggggagcctgctggagctggcagagctggcctgtgatggctacttagtgtctaccccacagcgtcctcccctctacctggaacgaattctctttgtcttactgcggaatgctgctgcacaaggaagcccagaggccacactccgccttgctcagcccctccatgcctgcttggtgcagtgctctcgcgaggctgctccccaggactatgaggccgtggctcggggcagcttttctctgctttggaagggggcagaagccctgttggaacggcgagctgcatttgcagctcggctgaaggccttgagcttcctagtactcttggaggatgaaagtaccccttgtgaggttcctcactttgcttctccaacagcctgtcgagcggtagctgcccatcagctatttgatgccagtggccatggtctaaatgaagcagatgctgatttcctagatgacctgctctccaggcacgtgatcagagccttggtgggtgagagagggagctcttctgggcttctttctccccagagggccctctgcctcttggagctcaccttggaacactgccgtcgcttttgctggagccgccaccatgacaaagccatcagcgcagtggagaaggctcacagttacctaaggaacaccaatctagcccctagccttcagctatgtcagctgggggttaagctgctgcaggtcggggaggaaggacctcaggcagtggccaagcttctgatcaaggcatcagctgtcctgagcaagagtatggaggcaccatcacccccacttcgggcattgtatgagagctgccagttcttcctttcaggcctggaacgaggcaccaagaggcgctatagacttgatgccattctgagcctctttgcttttcttggagggtactgctctcttctgcagcagctgcgggatgatggtgtgtatgggggctcctccaagcaacagcagtcttttcttcagatgtactttcagggacttcacctctacactgtggtggtttatgactttgcccaaggctgtcagatagttgatttggctgacctgacccaactagtggacagttgtaaatctaccgttgtctggatgctggaggccttagagggcctgtcgggccaagagctgacggaccacatggggatgaccgcttcttacaccagtaatttggcctacagcttctatagtcacaagctctatgccgaggcctgtgccatctctgagccgctctgtcagcacctgggtttggtgaagccaggcacttatcccgaggtgcctcctgagaagttgcacaggtgcttccggctacaagtagagagtttgaagaaactgggtaaacaggcccagggctgcaagatggtgattttgtggctggcagccctgcaaccctgtagccctgaacacatggctgagccagtcactttctgggttcgggtcaagatggatgcggccagggctggagacaaggagctacagctaaagactctgcgagacagcctcagtggctgggacccggagaccctggccctcctgctgagggaggagctgcaggcctacaaggcggtgcgggccgacactggacaggaacgcttcaacatcatctgtgacctcctggagctgagccccgaggagacaccagccggggcctgggcacgagccacccacctggtagaactggctcaggtgctctgctaccacgactttacgcagcagaccaactgctctgctctggatgctatccgggaagccctgcagcttctggactctgtgaggcctgaggcccaggccagagatcagcttctggacgataaagcacaggccttgctgtggctttacatctgtactctggaagccaaaatacaggaaggtatcgagcgggatcggagagcccaggcccctggtaacttggaggaatttgaagtcaatgacctgaactatgaagataaactccaggaagatcgtttcctatacagtaacattgccttcaacctggctgcagatgctgctcagtccaaatgcctggaccaagccctggccctgtggaaggagctgcttacaaaggggcaggccccagctgtacggtgtctccagcagacagcagcctcactgcagatcctagcagccctctaccagctggtggcaaagcccatgcaggctctggaggtcctcctgctgctacggattgtctctgagagactgaaggaccactcgaaggcagctggctcctcctgccacatcacccagctcctcctgaccctcggctgtcccagctatgcccagttacacctggaagaggcagcatcgagcctgaagcatctcgatcagactactgacacatacctgctcctttccctgacctgtgatctgcttcgaagtcaactctactggactcaccagaaggtgaccaagggtgtctctctgctgctgtctgtgcttcgggatcctgccctccagaagtcctccaaggcttggtacttgctgcgtgtccaggtcctgcagctggtggcagcttaccttagcctcccgtcaaacaacctctcacactccctgtgggagcagctctgtgcccaaggctggcagacacctgagatagctctcatagactcccataagctcctccgaagcatcatcctcctgctgatgggcagtgacattctctcaactcagaaagcagctgtggagacatcgtttttggactatggtgaaaatctggtacaaaaatggcaggttctttcagaggtgctgagctgctcagagaagctggtctgccacctgggccgcctgggtagtgtgagtgaagccaaggccttttgcttggaggccctaaaacttacaacaaagctgcagataccacgccagtgtgccctgttcctggtgctgaagggcgagctggagctggcccgcaatgacattgatctctgtcagtcggacctgcagcaggttctgttcttgcttgagtcttgcacagagtttggtggggtgactcagcacctggactctgtgaagaaggtccacctgcagaaggggaagcagcaggcccaggtcccctgtcctccacagctcccagaggaggagctcttcctaagaggccctgctctagagctggtggccactgtggccaaggagcctggccccatagcaccttctacaaactcctccccagtcttgaaaaccaagccccagcccatacccaacttcctgtcccattcacccacctgtgactgctcgctctgcgccagccctgtcctcacagcagtctgtctgcgctgggtattggtcacggcaggggtgaggctggccatgggccaccaagcccagggtctggatctgctgcaggtcgtgctgaagggctgtcctgaagccgctgagcgcctcacccaagctctccaagcttccctgaatcataaaacacccccctccttggttccaagcctcttggatgagatcttggctcaagcatacacactgttggcactggagggcctgaaccagccatcaaacgagagcctgcagaaggttctacagtcagggctgaagtttgtagcagcacggataccccacctagagccctggcgagccagcctgctcttgatttgggccctcacaaaactaggtggcctcagctgctgtactacccaactttttgcaagctcctggggctggcagccaccattaataaaaagtgtccctggctcagagccctctaagactcagggccaaaaacgttctggacgagggcgccaaaagttagcctctgctcccctgagcctcaataatacctctcagaaaggtctggaaggtagaggactgccctgcacacctaaacccccagaccggatcaggcaagctggccctcatgtccccttcacggtgtttgaggaagtctgccctacagagagcaagcctgaagtaccccaggcccccagggtacaacagagagtccagacgcgcctcaaggtgaacttcagtgatgacagtgacttggaagaccctgtctcagctgaggcctggctggcagaggagcctaagagacggggcactgcttcccggggccgggggcgagcaaggaagggcctgagcctaaagacggatgccgtggttgccccaggtagtgcccctgggaaccctggcctgaatggcaggagccggagggccaagaaggtggcatcaagacattgtgaggagcggcgtccccagagggccagtgaccaggccaggcctggccctgagatcatgaggaccatccctgaggaagaactgactgacaactggagaaaaatgagctttgagatcctcaggggctctgacggggaagactcagcctcaggtgggaagactccagctccgggccctgaggcagcttctggagaatgggagctgctgaggctggattccagcaagaagaagctgcccagcccatgcccagacaaggagagtgacaaggaccttggtcctcggctccagctcccctcagcccccgtagccactggtctttctaccctggactccatctgtgactccctgagtgttgctttccggggcattagtcactgtcctcctagtgggctctatgcccacctctgccgcttcctggccttgtgcctgggccaccgggatccttatgccactgctttccttgtcaccgagtctgtctccatcacctgtcgccaccagctgctcacccacctccacagacagctcagcaaggcccagaagcaccgaggatcacttgaaatagcagaccagctgcaggggctgagccttcaggagatgcctggagatgtccccctggcccgcatccagcgcctcttttccttcagggctttggaatctggccacttcccccagcctgaaaaggagagtttccaggagcgcctggctctgatccccagtggggtgactgtgtgtgtgttggccctggccaccctccagcccggaaccgtgggcaacaccctcctgctgacccggctggaaaaggacagtcccccagtcagtgtgcagattcccactggccagaacaagcttcatctgcgttcagtcctgaatgagtttgatgccatccagaaggcacagaaagagaacagcagctgtactgacaagcgagaatggtggacagggcggctggcactggaccacaggatggaggttctcatcgcttccctagagaagtctgtgctgggctgctggaaggggctgctgctgccgtccagtgaggagcccggccctgcccaggaggcctcccgcctacaggagctgctacaggactgtggctggaaatatcctgaccgcactctgctgaaaatcatgctcagtggtgccggtgccctcacccctcaggacattcaggccctggcctacgggctgtgcccaacccagccagagcgagcccaggagctcctgaatgaggcagtaggacgtctacagggcctgacagtaccaagcaatagccaccttgtcttggtcctagacaaggacttgcagaagctgccgtgggaaagcatgcccagcctccaagcactgcctgtcacccggctgccctccttccgcttcctactcagctactccatcatcaaagagtatggggcctcgccagtgctgagtcaaggggtggatccacgaagtaccttctatgtcctgaaccctcacaataacctgtcaagcacagaggagcaatttcgagccaatttcagcagtgaagctggctggagaggagtggttggggaggtgccaagacctgaacaggtgcaggaagccctgacaaagcatgatttgtatatctatgcagggcatggggctggtgcccgcttccttgatgggcaggctgtcctgcggctgagctgtcgggcagtggccctgctgtttggctgtagcagtgcggccctggctgtgcatggaaacctggagggggctggcatcgtgctcaagtacatcatggctggttgccccttgtttctgggtaatctctgggatgtgactgaccgcgacattgaccgctacacggaagctctgctgcaaggctggcttggagcaggcccaggggccccccttctctactatgtaaaccaggcccgccaagctccccgactcaagtatcttattggggctgcacctatagcctatggcttgcctgtctctctgcggtaa

SEQ ID NO: 2 - DNA, 6363 bp, Homo sapiens
atgaggagcttcaaaagagtcaactttgggactctgctaagcagccagaaggaggctgaagagttgctgcccgacttgaaggagttcctgtccaaccctccagctggttttcccagcagccgatctgatgctgagaggagacaagcttgtgatgccatcctgagggcttgcaaccagcagctgactgctaagctagcttgccctaggcatctggggagcctgctggagctggcagagctggcctgtgatggctacttagtgtctaccccacagcgtcctcccctctacctggaacgaattctctttgtcttactgcggaatgctgctgcacaaggaagcccagaggccacactccgccttgctcagcccctccatgcctgcttggtgcagtgctctcgcgaggctgctccccaggactatgaggccgtggctcggggcagcttttctctgctttggaagggggcagaagccctgttggaacggcgagctgcatttgcagctcggctgaaggccttgagcttcctagtactcttggaggatgaaagtaccccttgtgaggttcctcactttgcttctccaacagcctgtcgagcggtagctgcccatcagctatttgatgccagtggccatggtctaaatgaagcagatgctgatttcctagatgacctgctctccaggcacgtgatcagagccttggtgggtgagagagggagctcttctgggcttctttctccccagagggccctctgcctcttggagctcaccttggaacactgccgtcgcttttgctggagccgccaccatgacaaagccatcagcgcagtggagaaggctcacagttacctaaggaacaccaatctagcccctagccttcagctatgtcagctgggggttaagctgctgcaggtcggggaggaaggacctcaggcagtggccaagcttctgatcaaggcatcagctgtcctgagcaagagtatggaggcaccatcacccccacttcgggcattgtatgagagctgccagttcttcctttcaggcctggaacgaggcaccaagaggcgctatagacttgatgccattctgagcctctttgcttttcttggagggtactgctctcttctgcagcagctgcgggatgatggtgtgtatgggggctcctccaagcaacagcagtcttttcttcagatgtactttcagggacttcacctctacactgtggtggtttatgactttgcccaaggctgtcagatagttgatttggctgacctgacccaactagtggacagttgtaaatctaccgttgtctggatgctggaggccttagagggcctgtcgggccaagagctgacggaccacatggggatgaccgcttcttacaccagtaatttggcctacagcttctatagtcacaagctctatgccgaggcctgtgccatctctgagccgctctgtcagcacctgggtttggtgaagccaggcacttatcccgaggtgcctcctgagaagttgcacaggtgcttccggctacaagtagagagtttgaagaaactgggtaaacaggcccagggctgcaagatggtgattttgtggctggcagccctgcaaccctgtagccctgaacacatggctgagccagtcactttctgggttcgggtcaagatggatgcggccagggctggagacaaggagctacagctaaagactctgcgagacagcctcagtggctgggacccggagaccctggccctcctgctgagggaggagctgcaggcctacaaggcggtgcgggccgacactggacaggaacgcttcaacatcatctgtgacctcctggagctgagccccgaggagacaccagccggggcctgggcacgagccacccacctggtagaactggctcaggtgctctgctaccacgactttacgcagcagaccaactgctctgctctggatgctatccgggaagccctgcagcttctggactctgtgaggcctgaggcccaggccagagatcagcttctggacgataaagcacaggccttgctgtggctttacatctgtactctggaagccaaaatacaggaaggtatcgagcgggatcggagagcccaggcccctggtaacttggaggaatttgaagtcaatgacctgaactatgaagataaactccaggaagatcgtttcctatacagtaacattgccttcaacctggctgcagatgctgctcagtccaaatgcctggaccaagccctggccctgtggaaggagctgcttacaaaggggcaggccccagctgtacggtgtctccagcagacagcagcctcactgcagatcctagcagccctctaccagctggtggcaaagcccatgcaggctctggaggtcctcctgctgctacggattgtctctgagagactgaaggaccactcgaaggcagctggctcctcctgccacatcacccagctcctcctgaccctcggctgtcccagctatgcccagttacacctggaagaggcagcatcgagcctgaagcatctcgatcagactactgacacatacctgctcctttccctgacctgtgatctgcttcgaagtcaactctactggactcaccagaaggtgaccaagggtgtctctctgctgctgtctgtgcttcgggatcctgccctccagaagtcctccaaggcttggtacttgctgcgtgtccaggtcctgcagctggtggcagcttaccttagcctcccgtcaaacaacctctcacactccctgtgggagcagctctgtgcccaaggctggcagacacctgagatagctctcatagactcccataagctcctccgaagcatcatcctcctgctgatgggcagtgacattctctcaactcagaaagcagctgtggagacatcgtttttggactatggtgaaaatctggtacaaaaatggcaggttctttcagaggtgctgagctgctcagagaagctggtctgccacctgggccgcctgggtagtgtgagtgaagccaaggccttttgcttggaggccctaaaacttacaacaaagctgcagataccacgccagtgtgccctgttcctggtgctgaagggcgagctggagctggcccgcaatgacattgatctctgtcagtcggacctgcagcaggttctgttcttgcttgagtcttgcacagagtttggtggggtgactcagcacctggactctgtgaagaaggtccacctgcagaaggggaagcagcaggcccaggtcccctgtcctccacagctcccagaggaggagctcttcctaagaggccctgctctagagctggtggccactgtggccaaggagcctggccccatagcaccttctacaaactcctccccagtcttgaaaaccaagccccagcccatacccaacttcctgtcccattcacccacctgtgactgctcgctctgcgccagccctgtcctcacagcagtctgtctgcgctgggtattggtcacggcaggggtgaggctggccatgggccaccaagcccagggtctggatctgctgcaggtcgtgctgaagggctgtcctgaagccgctgagcgcctcacccaagctctccaagcttccctgaatcataaaacacccccctccttggttccaagcctcttggatgagatcttggctcaagcatacacactgttggcactggagggcctgaaccagccatcaaacgagagcctgcagaaggttctacagtcagggctgaagtttgtagcagcacggataccccacctagagccctggcgagccagcctgctcttgatttgggccctcacaaaactaggtggcctcagctgctgtactacccaactttttgcaagctcctggggctggcagccaccattaataaaaagtgtccctggctcagagccctctaagactcagggccaaaaacgttctggacgagggcgccaaaagttagcctctgctcccctgagcctcaataatacctctcagaaaggtctggaaggtagaggactgccctgcacacctaaacccccagaccggatcaggcaagctggccctcatgtccccttcacggtgtttgaggaagtctgccctacagagagcaagcctgaagtaccccaggcccccagggtacaacagagagtccagacgcgcctcaaggtgaacttcagtgatgacagtgacttggaagaccctgtctcagctgaggcctggctggcagaggagcctaagagacggggcactgcttcccggggccgggggcgagcaaggaagggcctgagcctaaagacggatgccgtggttgccccaggtagtgcccctgggaaccctggcctgaatggcaggagccggagggccaagaaggtggcatcaagacattgtgaggagcggcgtccccagagggccagtgaccaggccaggcctggccctgagatcatgaggaccatccctgaggaagaactgactgacaactggagaaaaatgagctttgagatcctcaggggctctgacggggaagactcagcctcaggtgggaagactccagctccgggccctgaggcagcttctggagaatgggagctgctgaggctggattccagcaagaagaagctgcccagcccatgcccagacaaggagagtgacaaggaccttggtcctcggctccagctcccctcagcccccgtagccactggtctttctaccctggactccatctgtgactccctgagtgttgctttccggggcattagtcactgtcctcctagtgggctctatgcccacctctgccgcttcctggccttgtgcctgggccaccgggatccttatgccactgctttccttgtcaccgagtctgtctccatcacctgtcgccaccagctgctcacccacctccacagacagctcagcaaggcccagaagcaccgaggatcacttgaaatagcagaccagctgcaggggctgagccttcaggagatgcctggagatgtccccctggcccgcatccagcgcctcttttccttcagggctttggaatctggccacttcccccagcctgaaaaggagagtttccaggagcgcctggctctgatccccagtggggtgactgtgtgtgtgttggccctggccaccctccagcccggaaccgtgggcaacaccctcctgctgacccggctggaaaaggacagtcccccagtcagtgtgcagattcccactggccagaacaagcttcatctgcgttcagtcctgaatgagtttgatgccatccagaaggcacagaaagagaacagcagctgtactgacaagcgagaatggtggacagggcggctggcactggaccacaggatggaggttctcatcgcttccctagagaagtctgtgctgggctgctggaaggggctgctgctgccgtccagtgaggagcccggccctgcccaggaggcctcccgcctacaggagctgctacaggactgtggctggaaatatcctgaccgcactctgctgaaaatcatgctcagtggtgccggtgccctcacccctcaggacattcaggccctggcctacgggctgtgcccaacccagccagagcgagcccaggagctcctgaatgaggcagtaggacgtctacagggcctgacagtaccaagcaatagccaccttgtcttggtcctagacaaggacttgcagaagctgccgtgggaaagcatgcccagcctccaagcactgcctgtcacccggctgccctccttccgcttcctactcagctactccatcatcaaagagtatggggcctcgccagtgctgagtcaaggggtggatccacgaagtaccttctatgtcctgaaccctcacaataacctgtcaagcacagaggagcaatttcgagccaatttcagcagtgaagctggctggagaggagtggttggggaggtgccaagacctgaacaggtgcaggaagccctgacaaagcatgatttgtatatctatgcagggcatggggctggtgcccgcttccttgatgggcaggctgtcctgcggctgagctgtcgggcagtggccctgctgtttggctgtagcagtgcggccctggctgtgcatggaaacctggagggggctggcatcgtgctcaagtacatcatggctggttgccccttgtttctgggtaatctctgggatgtgactgaccgcgacattgaccgctacacggaagctctgctgcaaggctggcttggagcaggcccaggggccccccttctctactatgtaaaccaggcccgccaagctccccgactcaagtatcttattggggctgcacctatagcctatggcttgcctgtctctctgcggtaa

SEQ ID NO: 3 - protein, 2120 aa, Homo sapiens
MRSFKRVNFGTLLSSQKEAEELLPDLKEFLSNPPAGFPSSRSDAERRQACDAILRACNQQLTAKLACPRHLGSLLELAELACDGYLVSTPQRPPLYLERILFVLLRNAAAQGSPEATLRLAQPLHACLVQCSREAAPQDYEAVARGSFSLLWKGAEALLERRAAFAARLKALSFLVLLEDESTPCEVPHFASPTACRAVAAHQLFDASGHGLNEADADFLDDLLSRHVIRALVGERGSSSGLLSPQRALCLLELTLEHCRRFCWSRHHDKAISAVEKAHSYLRNTNLAPSLQLCQLGVKLLQVGEEGPQAVAKLLIKASAVLSKSMEAPSPPLRALYESCQFFLSGLERGTKRRYRLDAILSLFAFLGGYCSLLQQLRDDGVYGGSSKQQQSFLQMYFQGLHLYTVVVYDFAQGCQIVDLADLTQLVDSCKSTVVWMLEALEGLSGQELTDHMGMTASYTSNLAYSFYSHKLYAEACAISEPLCQHLGLVKPGTYPEVPPEKLHRCFRLQVESLKKLGKQAQGCKMVILWLAALQPCSPEHMAEPVTFWVRVKMDAARAGDKELQLKTLRDSLSGWDPETLALLLREELQAYKAVRADTGQERFNIICDLLELSPEETPAGAWARATHLVELAQVLCYHDFTQQTNCSALDAIREALQLLDSVRPEAQARDQLLDDKAQALLWLYICTLEAKIQEGIERDRRAQAPGNLEEFEVNDLNYEDKLQEDRFLYSNIAFNLAADAAQSKCLDQALALWKELLTKGQAPAVRCLQQTAASLQILAALYQLVAKPMQALEVLLLLRIVSERLKDHSKAAGSSCHITQLLLTLGCPSYAQLHLEEAASSLKHLDQTTDTYLLLSLTCDLLRSQLYWTHQKVTKGVSLLLSVLRDPALQKSSKAWYLLRVQVLQLVAAYLSLPSNNLSHSLWEQLCAQGWQTPEIALIDSHKLLRSIILLLMGSDILSTQKAAVETSFLDYGENLVQKWQVLSEVLSCSEKLVCHLGRLGSVSEAKAFCLEALKLTTKLQIPRQCALFLVLKGELELARNDIDLCQSDLQQVLFLLESCTEFGGVTQHLDSVKKVHLQKGKQQAQVPCPPQLPEEELFLRGPALELVATVAKEPGPIAPSTNSSPVLKTKPQPIPNFLSHSPTCDCSLCASPVLTAVCLRWVLVTAGVRLAMGHQAQGLDLLQVVLKGCPEAAERLTQALQASLNHKTPPSLVPSLLDEILAQAYTLLALEGLNQPSNESLQKVLQSGLKFVAARIPHLEPWRASLLLIWALTKLGGLSCCTTQLFASSWGWQPPLIKSVPGSEPSKTQGQKRSGRGRQKLASAPLSLNNTSQKGLEGRGLPCTPKPPDRIRQAGPHVPFTVFEEVCPTESKPEVPQAPRVQQRVQTRLKVNFSDDSDLEDPVSAEAWLAEEPKRRGTASRGRGRARKGLSLKTDAVVAPGSAPGNPGLNGRSRRAKKVASRHCEERRPQRASDQARPGPEIMRTIPEEELTDNWRKMSFEILRGSDGEDSASGGKTPAPGPEAASGEWELLRLDSSKKKLPSPCPDKESDKDLGPRLQLPSAPVATGLSTLDSICDSLSVAFRGISHCPPSGLYAHLCRFLALCLGHRDPYATAFLVTESVSITCRHQLLTHLHRQLSKAQKHRGSLEIADQLQGLSLQEMPGDVPLARIQRLFSFRALESGHFPQPEKESFQERLALIPSGVTVCVLALATLQPGTVGNTLLLTRLEKDSPPVSVQIPTGQNKLHLRSVLNEFDAIQKAQKENSSCTDKREWWTGRLALDHRMEVLIASLEKSVLGCWKGLLLPSSEEPGPAQEASRLQELLQDCGWKYPDRTLLKIMLSGAGALTPQDIQALAYGLCPTQPERAQELLNEAVGRLQGLTVPSNSHLVLVLDKDLQKLPWESMPSLQALPVTRLPSFRFLLSYSIIKEYGASPVLSQGVDPRSTFYVLNPHNNLSSTEEQFRANFSSEAGWRGVVGEVPRPEQVQEALTKHDLYIYAGHGAGARFLDGQAVLRLSCRAVALLFGCSSAALAVHGNLEGAGIVLKYIMAGCPLFLGNLWDVTDRDIDRYTEALLQGWLGAGPGAPLLYYVNQARQAPRLKYLIGAAPIAYGLPVSLR

SEQ ID NO: 4 - DNA, 32 bp, artificial sequence
  amplification primer
atgaggagcttcaaaagagtcaactttgggac

SEQ ID NO: 5 - DNA, 24 bp, artificial sequence
  amplification primer
ttaccgcagagagacaggcaagcc

SEQ ID NO: 6 - protein, 15 aa, artificial sequence
  peptide to raise antibodies
RSFKRVNFGTLLSSQ

SEQ ID NO: 7 - protein, 15 aa, artificial sequence
  peptide to raise antibodies
EPYSDIIATPGPRFH

SEQ ID NO: 8 - DNA, 26 bp, artificial sequence
  amplification primer
atgttctacgcacattttgttctcag

SEQ ID NO: 9 - DNA, 26 bp, artificial sequence
  amplification primer
tataatatggaaccttggtccaggtg

(19) United States
(12) Patent Application Publication
(10) Pub. No.: US 2003/0148462 A1
(43) Pub. Date: Aug. 7, 2003

(54) Dual inhibition of sister chromatid separation at metaphase

(75) Inventors: Marc W. Kirschner, Newton, MA (US); Olaf Stemmann, Munich (DE); Hui Zou, Dallas, TX (US); Steven P. Gygi, Foxborough, MA (US)

Correspondence Address: BANNER & WITCOFF, LTD., 28 STATE STREET, 28th FLOOR, BOSTON, MA, 02109-9601, US

(73) Assignee: President and fellows of Harvard College, Cambridge, MA (US)

(21) Appl. No.: 10/320,175

(22) Filed: Dec. 16, 2002

Related U.S. Application Data
(60) Provisional application No. 60/340,682, filed on Dec. 14, 2001.

(51) Int. Cl. C07H 21/04; C12N 9/22; C12N 9/24; C12P 21/02; C12N 5/06

(52) U.S. Cl. 435/69.1; 435/200; 435/320.1; 435/325; 435/199; 536/23.2

(57) ABSTRACT

The invention provides nucleic acid molecules, designated separase nucleic acid molecules, which encode separase, an endopeptidase that modulates sister chromatid separation. The invention also provides recombinant expression vectors containing separase nucleic acid molecules and host cells into which the expression vectors have been introduced. The invention still further provides separase proteins, fusion proteins, antigenic peptides and anti-separase antibodies. The invention also provides methods for the identification of modulators of separase, methods of modulating separase, methods of modulating sister chromatid separation, and methods for the treatment of disorders related to aberrant sister chromatid separation, such as cancer, Down's syndrome, and spontaneous fetal abortion.

### RELATED APPLICATIONS

This application claims priority to U.S. Provisional Patent Application No. 60/340,682, filed on Dec. 14, 2001, hereby incorporated by reference in its entirety for all purposes.

### STATEMENT OF GOVERNMENT INTERESTS

This invention was made with government support under grant numbers HG00041, GM26875-17, and GM39023-08, awarded by the National Institutes of Health. The Government has certain rights in the invention.

### BACKGROUND

Sister chromatid cohesion is mediated by a multi-protein complex, cohesin (Guacci et al. (1997) Cell 91:47; Michaelis et al. (1997) Cell 91:35). In vertebrates, the majority of cohesin dissociates from chromosomes at prophase (Losada et al. (1998) Genes Dev. 12:1986). Nonetheless, sister chromatid cohesion is maintained in centromeric regions by remaining cohesin complexes (Waizenegger et al. (2000) Cell 103:399). At the metaphase to anaphase transition, residual cohesin complexes are removed via the cleavage of the cohesin subunit SCC1 by a cysteine endopeptidase, separase. This cleavage is both sufficient and necessary for the separation of sister chromatids (Uhlmann et al. (1999) Nature 400:37; Uhlmann et al. (2000) Cell 103:375; Waizenegger et al. (2000) Cell 103:399).

The timing of sister chromatid separation is linked to the mitotic cell cycle by the destruction of an anaphase inhibitor, securin. Securin was identified in yeast (Yamamoto et al. (1996) J. Cell Biol. 133:85) and its functional homologues, which are widely divergent in sequence, were later found in higher eukaryotes (Zou et al. (1999) Science 285, 418; Leismann et al. (2000) Genes Dev. 14:2192). Before anaphase, securin forms a complex with separase and presumably inhibits its activity (Ciosk et al. (1998) Cell 93:1067; Zou et al. (1999) Science 285, 418). At anaphase, securin is degraded by ubiquitin-dependent proteolysis mediated by the anaphase promoting complex (APC) (for review, see King et al. (1996a) Science 274:1652). This proteolysis pathway is under the control of the mitotic spindle checkpoint, which ties the separation of sister chromatids to the successful assembly of the mitotic spindle.

### SUMMARY OF THE INVENTION

Embodiments of the present invention relate to the identification and characterization of a human cysteine endopeptidase protein involved in the regulation of the control of sister chromatid separation, referred to herein as “separase.” The separase molecules of the present invention are useful as modulating agents to regulate the separation of sister chromatids and to modulate or otherwise regulate cellular processes related to sister chromatid separation. The separase nucleic acids and polypeptides of the present invention are useful for both in vitro and in vivo modulation of sister chromatid separation, as well as for the treatment of disorders associated with aberrant sister chromatid separation such as cancer, Down's syndrome, spontaneous fetal abortion.

Accordingly, embodiments of the present invention are directed to nucleic acid molecules and polypeptides encoding separase, i.e., separase nucleic acids, protein molecules, and their analogs. In particular, the present invention is directed to methods of detecting nucleic acids and polypeptides that encode separase in samples, methods of detecting separase phosphorylation, methods of modulating separase activity (e.g., modulating cohesin^{hSCC1} cleavage, separase cleavage, and sister chromatid separation), and methods of identifying modulators of separase activity. The present invention also features separase nucleic acid molecules that specifically detect separase nucleic acid molecules relative to non-separase nucleic acid molecules.

Embodiments of the present invention also relate to vectors encoding separase nucleic acid molecules, such as recombinant expression vectors. Vectors encoding separase nucleic acids can be provided in host cells. Accordingly, the present invention provides methods for producing separase nucleic acids and polypeptides by culturing a host cell containing a recombinant expression vector in a suitable medium to produce separase nucleic acids and polypeptides.

The separase polypeptides of the present invention or biologically active portions thereof, can be operatively linked to a non-separase polypeptide (e.g., heterologous amino acid sequences) to form fusion proteins. Embodiments of the present invention further include antibodies, such as monoclonal or polyclonal antibodies, that specifically bind phosphorylated or unphosphorylated separase polypeptides of the invention. In addition, the separase polypeptides or biologically active portions thereof can be incorporated into pharmaceutical compositions, which optionally include pharmaceutically acceptable carriers.

Embodiments of the present invention further provide methods for modulating separase activity. Such methods include contacting a separase nucleic acid, a separase polypeptide, a cell capable of expressing a separase nucleic acid or polypeptide, or a subject, with an agent that modulates separase activity. Modulating separase with a compound can be useful for increasing or decreasing sister chromatid separation. Embodiments of the present invention also provide methods for treating a disorder in a subject by modulating separase activity. Compounds of the present invention can inhibit separase activity (e.g., by phosphorylating separase), or stimulate separase activity (e.g., by dephosphorylating separase). Useful compounds include antibodies that specifically bind to a separase protein, compounds that increase or decrease expression of separase by modulating transcription of a separase gene or translation of a separase mRNA, and nucleic acid molecules having a nucleotide sequence that is antisense to the coding strand of a separase mRNA or a separase gene. Separase modulators of the present invention can include separase polypeptides, separase nucleic acid molecules, peptides, peptidomimetics, or other small molecules.

Other features and advantages of the invention will be apparent from the following detailed description and claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present invention will be more fully understood from the following detailed description of illustrative embodiments taken in conjunction with the accompanying drawings in which:

FIGS. 1A-1C illustrate that high CDC2/cyclinB1 activity inhibits sister chromatid separation and segregation in Xenopus extracts but not securin degradation. (A) depicts the effects of non-degradable cyclinB1 (Δ90) and the CDC2 inhibitor roscovitine on anaphase and mitotic exit. (B) depicts a histone H1 kinase assay for selected extracts as used in (A). (C) depicts ³⁵S-labeled Xenopus securin and an N-terminal fragment of cyclinB1 were generated by in vitro translation and added to CSF-extracts. The kinetics of securin degradation after Ca²⁺ addition was measured in the presence (500 nM) or absence of human Δ90 (upper panel). In the lower panel, degradation of cyclinB1 was detected 45 minutes after Ca²⁺ addition. The extract contained 32 to 500 nM human Δ90 (lanes 2 to 6; twofold increase in concentration between each lane), 82 to 1300 nM sea urchin Δ90 (lanes 7 to 11), or 50 to 800 nM unlabeled cyclinB1 fragment (lanes 12 to 16). Lane1: Negative control without Ca²⁺ addition.

FIGS. 2A-2B illustrate the inhibition of separase activity by high-Δ90 extracts. (A) depicts an in vitro separase activity assay. Tagged separase and associated securin were affinity-purified from nocodazole-arrested 293T cells. The left panel shows Western blots of isolated securin/separase complexes before (lanes 1 and 2) and after (lanes 3 and 4) incubation with low-Δ90 extract. Separase was re-isolated, eluted, and assayed for cohesin^{hSCC1} cleavage activity. In vitro translated, radiolabeled cohesin^{hSCC1} (lanes 5 and 6) or endogenous cohesin^{hSCC1} on purified metaphase chromosomes (lanes 7 and 8) served as substrates. Cohesin^{hSCC1} and its cleavage fragments were detected by autoradiography or anti-cohesin^{hSCC1} immunoblot, respectively. The assay was performed with wild type separase (WT; lanes 1, 3, 5, 7) and a catalytically inactive separase mutant (C [symbol] S; lanes 2, 4, 6, 8). (B) depicts Western blots with anti-separase (upper left panel), anti-securin (lower left panel), and anti-cohesin^{hSCC1} (right panel) antibodies.

FIGS. 3A-3D depict purification of the securin/separase-complex from nocodazole-arrested HeLaS3 cells. (A) depicts a purification scheme and chromatograph of the final purification step (a bracket indicates the elution position of the securin/separase complex (fractions 5 and 6)). (B) depicts Western blots using both anti-separase (upper panel) and anti-securin (lower panel) antibodies. (C) depicts silver staining of the proteins in Mini Q fractions 1 to 9. (D) depicts a separase activity assay. In this modified assay, 2 μl of each Mini Q fraction were combined with 10 μl of a low-Δ90 extract and 2 μl of in vitro translated ³⁵S-cohesin^{hSCC1}. After incubation for 1 hour at room temperature and 1 hour at 37° C., 2 μl of each reaction were analyzed by SDS-PAGE and autoradiography. Molecular weights in kDa are marked on the left side of the pictures shown in (A), (B), and (C).

FIGS. 4A-4B depict separase inhibition by direct phosphorylation at one major site. (A) depicts mass spectrometric determination of phosphorylation sites on separase. The relative positions of the mapped sites on separase are illustrated on the left side. These sites correspond to Ser1073, -1126, -1305, -1501, -1508, -1545, -1552, and Thr1346. Shown on the right is the tandem mass (MS/MS) spectrum of a phospho-peptide derived by collision-induced dissociation of the (M+2H)²⁺ precursor, m/z 724. (B) depicts the functional identification of the inhibitory phosphorylation site(s). Mutant separases (PMs), which had the serine and/or threonine sites changed to alanine, were analyzed by the separase activity assay. Numbers indicate which phosphorylation site(s) were changed in each PM mutant.

FIGS. 5A-5B illustrate that sister chromatid separation in high-Δ90 extract can be rescued by a single point mutation in separase. (A) depicts re-isolated chromosomes that were stained with DAPI and CREST serum (stained center of chromatid) and analyzed by fluorescence microscopy. (B) depicts an anti-separase Western blot. The amounts of separase used in the sister chromatid separation assay (A) were compared to each other by immuno-blotting.

FIGS. 6A-6E illustrate that the inhibitory phosphorylation of separase is high in metaphase and declines upon anaphase onset. (A) depicts FACS and Western analyses of synchronized HeLaS3 cells undergoing mitosis. (B) depicts quantification of cell cycle distribution and phosphorylation status of separase at Ser1126 for the samples shown in (A). (C) depicts a nano-scale microcapillary LC-MS/MS analysis of native separase phosphorylation state. Shown are the selected reaction, extracted-ion chromatograms corresponding to the unphosphorylated (upper trace) and phosphorylated (bottom trace) Glu1115-Lys1130 native tryptic peptide (blue and green) and heavy internal standard (brown and red). Inset: Averaged selected reaction m/z window corresponding to the y₁₀-ion fragment of light and heavy peptides. (D) depicts Ser1136-specific in vitro kinase assays. Shown is the phosphorylation status of Ser1126 in percent as determined by incubation of affinity-purified securin/separase with various pure kinases in the presence of ATP (1 mM) followed by LC-MS/MS analysis. (E) depicts a LC-MS/MS result for CDC2/cyclinB1. Top panel: Mock treatment. Bottom panel: CDC2/cyclinB1. 88% of separase was phosphorylated.

FIGS. 7A-7C depict the independent inhibition of separase activity by phosphorylation of separase and by binding of securin. (A) depicts a Western blot (upper panel) and cohesin^{hSCC1} cleavage activity using isolated chromosomes as substrate (lower panel). Lane 2: Consecutive treatment of separase with high-Δ90 extract twice. (B) depicts separase that had been pre-activated in low-Δ90 extract that was eluted and incubated with recombinant securin (lanes 2 and 3) or reference buffer (lane 1) for one hour on ice. Subsequently, its cleavage activity towards in vitro translated ³⁵S-cohesin^{hSCC1} was tested (upper panel). The separase concentration in each reaction was 4 nM, as estimated by Coomassie staining. To compare wild type separase with PM-2/4 mutant separase in its ability to bind securin the same experiment was repeated with PM-2/4 (lower panel). Roughly equal separase concentrations in both cases were assured by comparative immunoblotting (see FIG. 4B, lanes 15 and 17). To compare wild type separase with PM-2/4 mutant separase in its ability to bind securin the same experiment was repeated with PM-2/4 (lower panel). (C) depicts a model for the dual inhibition of separase in metaphase and its activation at anaphase onset. PPase denotes an unknown protein phosphatase that is proposed to act on phosphorylated separase.

FIG. 8 depicts the nucleotide sequence of the open reading frame of human separase mRNA including a potential unspliced intron (set forth as SEQ ID NO:1).

FIG. 9 depicts the nucleotide sequence of the open reading frame of human separase mRNA (set forth as SEQ ID NO:2).

FIG. 10 depicts the amino acid sequence of the human separase protein (set forth as SEQ ID NO:3).

### DETAILED DESCRIPTION

Embodiments of the present invention relates to the isolation and characterization of a human cysteine endopeptidase protein involved in the regulation and/or inhibition of the control of sister chromatid separation, referred to herein as “separase.” The present invention is further based on the discovery that phosphorylation and dephosphorylation of separase will regulate, i.e. inhibit or promote, the separation of sister chromatids. Embodiments of the present invention are thus directed to the regulation of separase for the temporal control of sister chromatid separation.

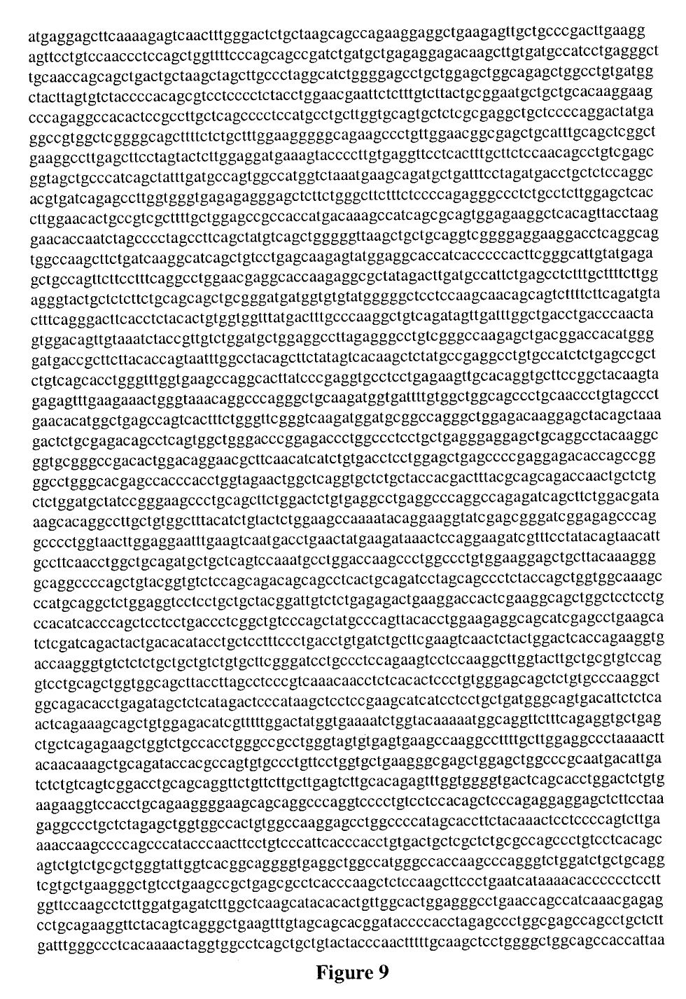

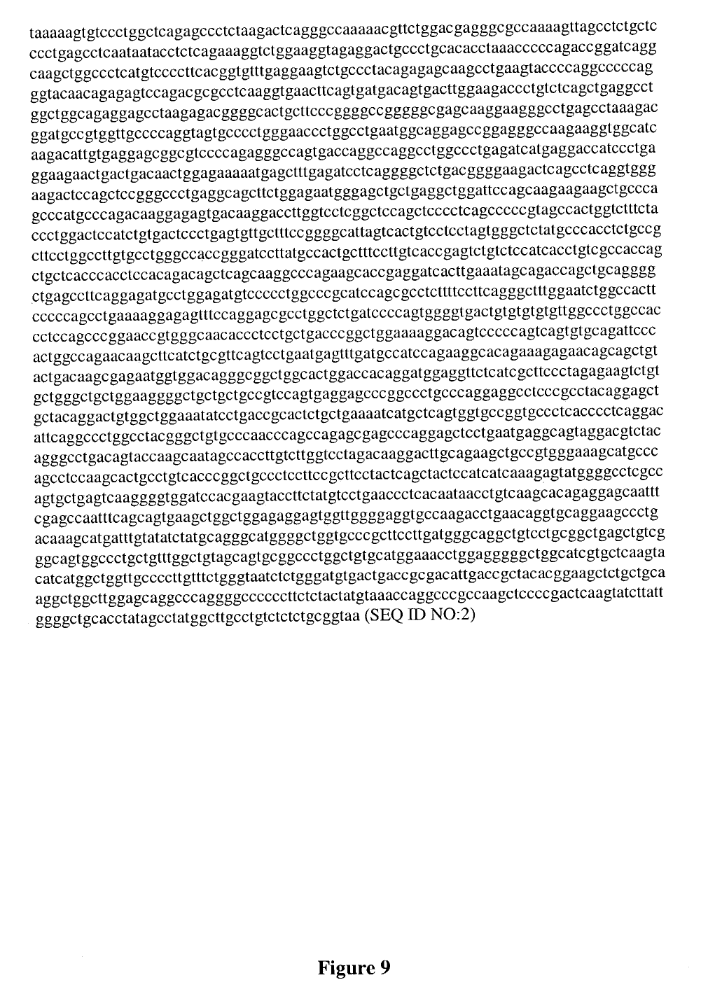

The human separase open reading frame sequence (set forth in FIG. 9; SEQ ID NO: 2), which is approximately 6,363 nucleotide residues long, contains a methionine-initiated coding sequence of about 2,120 nucleotide residues, excluding the termination codon (i.e., nucleotide residues 1-6,360 of SEQ ID NO: 2; also shown in SEQ ID NO: 3).

In one embodiment, a separase nucleic acid molecule of the invention is at least 40%, 50%, 60%, 65%, 70%, 75%, 80%, 85%, 86%, 87%, 88%, 89%, 90%, 91%, 92%, 93%, 94%, 95%, 96%, 97%, 98%, 98.9%, 99.0%, 99.1%, 99.2%, 99.3%, 99.4%, 99.5%, 99.6%, 99.7%, 99.8%, 99.9% or more identical to the nucleotide sequence (e.g., to the entire length of the nucleotide sequence) shown in SEQ ID NO:2.

In another preferred embodiment, the nucleic acid molecule includes the nucleotide sequence shown in SEQ ID NO:2, or complements and/or analogs thereof.

In another embodiment, a separase nucleic acid molecule includes a nucleotide sequence encoding a protein having an amino acid sequence sufficiently identical to the amino acid sequence of SEQ ID NO:3. In a preferred embodiment, a separase nucleic acid molecule includes a nucleotide sequence encoding a protein having an amino acid sequence at least 40%, 50%, 60%, 65%, 70%, 75%, 80%, 85%, 86%, 87%, 88%, 89%, 90%, 91%, 92%, 93%, 94%, 95%, 96%, 97%, 98%, 99% or more identical to the amino acid sequence of SEQ ID NO:3.

The present invention also features nucleic acid molecules, preferably separase nucleic acid molecules and analogs thereof, which specifically detect separase nucleic acid molecules relative to nucleic acid molecules encoding non-separase proteins. For example, in one embodiment, such a nucleic acid molecule is at least 15, 30, 50, 100, 250, 500, 1000, 1500, 2000, 2500, 3000, 3500, 4000, 4500, 5000, 5500, or 6000 or more nucleotides in length and hybridizes, preferably under stringent conditions, to a nucleic acid molecule comprising the nucleotide sequence shown in SEQ ID NO:2.

In other preferred embodiments, the nucleic acid molecule encodes a naturally occurring allelic variant of a polypeptide comprising the amino acid sequence of SEQ ID NO:3, wherein the nucleic acid molecule hybridizes to a nucleic acid molecule comprising SEQ ID NO:2, preferably, under stringent conditions.

As used herein, the term “hybridizes under stringent conditions” is intended to describe conditions for hybridization and washing under which nucleotide sequences at least 60% identical to each other typically remain hybridized to each other. Preferably, the conditions are such that sequences at least about 70%, more preferably at least about 80%, even more preferably at least about 85% or 90% identical to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6×sodium chloride/sodium citrate (SSC) at about 45° C., followed by one or more washes in 0.2×SSC, 0.1% SDS at 50° C., preferably at 55° C., and more preferably at 60° C. or 65° C. Preferably, a nucleic acid molecule of the invention that hybridizes under stringent conditions to the sequence of SEQ ID NO:2 corresponds to a naturally-occurring nucleic acid molecule. As used herein, the term “analog” includes an RNA or DNA molecule that can be identified using these stringent hybridization conditions as well as amino acids and polypeptides encoded by the RNA or DNA so identified. As used herein, a “naturally-occurring” nucleic acid molecule refers to an RNA or DNA molecule having a nucleotide sequence that occurs in nature (e.g., encodes a natural protein).

Another embodiment of the invention provides a nucleic acid molecule which is antisense to a separase nucleic acid molecule, e.g., the coding strand of a separase nucleic acid molecule.

Human separase contains eight predicted phosphorylation sites at about amino acid residues S1073, S1126, S1305, T1346, S1501, S1508, S1545 and S1552 of SEQ ID NO: 3. Human separase also contains a catalytic cysteine residue at about cysteine 2029 of SEQ ID NO:3. Human separase also contains autocatalytic cleavage sites at least at about amino acids R1486, R1506, and R1535 of SEQ ID NO:3. Cleavage of separase results in the generation of two fragments that migrate at approximately 175 kDa and 55 kDa.

Another embodiment of the invention features isolated or recombinant separase proteins and polypeptides. In one embodiment, separase includes at least one phosphorylation site, and more preferably two, three, four, five, six, seven, or eight phosphorylation sites. In another embodiment, separase contains at least one catalytic cysteine residue. In another embodiment, separase includes at least one autocatalytic cleavage site, and more preferably two or three autocatalytic cleavage sites. In a preferred embodiment, a separase polypeptide or separase analog includes at least one phosphorylation site and has an amino acid sequence at least about 40%, 50%, 60%, 65%, 70%, 75%, 80%, 85%, 86%, 87%, 88%, 89%, 90%, 91%, 92%, 92%, 93%, 94%, 95%, 96%, 97%, 98%, 99% or more identical to the amino acid sequence of SEQ ID NO:3, or the amino acid sequence encoded by a nucleic acid molecule having the nucleic acid sequence of SEQ ID NO:2. In another preferred embodiment, separase includes at least one phosphorylation site and modulates sister chromatid separation. In yet another preferred embodiment, separase includes at least one phosphorylation site and is encoded by a nucleic acid molecule having a nucleotide sequence which hybridizes under stringent hybridization conditions to a nucleic acid molecule comprising the nucleotide sequence of SEQ ID NO:2.

In another embodiment, the invention features fragments of the proteins having the amino acid sequence of SEQ ID NO:3, wherein the fragment comprises at least 10 amino acids (e.g., contiguous amino acids) of the N-terminal 325 amino acids of the amino acid sequence of SEQ ID NO:3. In another embodiment, the invention features fragments of the proteins having the amino acid sequence of SEQ ID NO:3, wherein the fragment comprises at least 1,796 amino acids (e.g., contiguous amino acids) of the amino acid sequence of SEQ ID NO:3. In another embodiment, separase has the amino acid sequence of SEQ ID NO:3.

In another embodiment, the invention features an isolated separase which is encoded by a nucleic acid molecule having a nucleotide sequence at least about 40%, 50%, 60%, 65%, 70%, 75%, 80%, 85%, 86%, 87%, 88%, 89%, 90%, 91%, 92%, 93%, 94%, 95%, 96%, 97%, 98%, 98.9%, 99.0%, 99.1%, 99.2%, 99.3%, 99.4%, 99.5%, 99.6%, 99.7%, 99.8%, 99.9% or more identical to a nucleotide sequence of SEQ ID NO:2, or a complement thereof.

As used herein, a “separase activity,” “biological activity of separase,” or “functional activity of separase,” refers to an activity exerted by a separase protein, polypeptide or nucleic acid molecule on, for example, a separase-responsive cell or on a separase substrate (e.g., cohesin^{hSCC1}) as determined in vivo or in vitro. In one embodiment, a separase activity is a direct activity, such as association with a separase target molecule. A “target molecule” or “binding partner” of separase is a molecule with which separase binds or interacts in nature (e.g., securin, cohesin^{hSCC1}, and the like). A separase activity can also be an indirect activity, such as sister chromatid separation mediated by interaction of separase with a separase target molecule or by de-phosphorylation of separase.

The separase proteins of the present invention can have one or more of the following activities: (1) catalyzing autocatalytic cleavage (i.e., self-cleavage of separase); (2) catalyzing cleavage of cohesin^{hSCC1}; (3) modulating sister chromatid separation; (4) modulating progression of a cell through the cell cycle; (5) modulating entry of a cell into the cell cycle; (6) modulating cell growth; (7) modulating tumorigenesis; and (8) modulating mitogenesis.

As used herein, the term “modulate” refers to a stimulation or inhibition of an activity, such as regulation of separase phosphorylation, separase cleavage, cohesin^{SCC1} cleavage, or sister chromatid separation. As used herein, the term “sister chromatid separation” refers to the simultaneous separation of sister chromatids and their migration to opposite spindle poles that occurs during cell division. As used herein, the term “cleavage” refers to the proteolytic cleavage of a polypeptide at one or more cleavage site. Cleavage may be autologous (e.g., self-cleavage of separase) or mediated by a separate protein (e.g., the cleavage of cohesin^{SCC1} by separase).

As used herein, the terms “inhibit” and “inhibition” refer to a partial inhibition or a complete inhibition of an activity, such as an inhibition of separase cleavage, cohesin^{SCC1} cleavage, or sister chromatid separation, or an inhibition of a disorder, disease, or condition such that therapeutic treatment and/or prophylaxis results. An inhibition of separase cleavage occurs, for example, when a cell expressing separase is contacted with a compound that inhibits and has a lower level of separase cleavage as compared to a cell expressing separase that is not contacted with the compound. A complete inhibition occurs, for example, when no separase cleavage is observed when separase is contacted with the compound as compared to when separase is not contacted with the compound. A partial inhibition of separase cleavage occurs, for example, when separase cleavage is observed in the presence of a compound, but at lower levels than in the absence of the compound. For example, separase cleavage may be reduced to 99%, 95%, 90%, 85%, 80%, 75%, 70%, 65%, 60%, 55%, 50%, 45%, 40%, 35%, 30%, 25%, 20%, 15%, 10%, 5%, or 1% of the level of separase cleavage in the absence of the compound.

As used herein, the terms “stimulate” and “stimulation” refer to an increase in an activity, such as an increase of separase cleavage, cohesin^{SCC1} cleavage, or sister chromatid separation, or a worsening of a disorder, disease, or condition. A stimulation in separase cleavage is observed, for example, when a cell expressing separase is contacted with a compound that stimulates and has a higher level of separase cleavage as compared to a cell expressing separase that is not contacted with a compound. A stimulation in separase cleavage occurs, for example, when separase cleavage is observed at least at 101%, 102%, 103%, 104%, 105%, 106%, 107%, 108%, 109%, 110%, 115%, 120%, 125%, 130%, 135%, 140%, 145%, 150%, 160%, 170%, 180%, 185%, 190%, 200%, 250%, 300%, 350%, 400%, 500%, 600%, 700%, 800%, 900%, or higher levels when compared to the levels of separase cleavage observed in a cell not contacted with a compound that stimulates separase cleavage.

Thus, the separase molecules described herein can act as novel diagnostic targets and therapeutic agents for the prognosis, diagnosis, prevention, inhibition, alleviation, or cure of disorders related to aberrant sister chromatid separation.

Screening Assays

The invention provides a method (also referred to herein as a “screening assay”) for identifying modulators, i.e., candidate or test compounds or agents (e.g., peptides, cyclic peptides, peptidomimetics, small molecules, small organic molecules, or other drugs) which bind to separase proteins, have a stimulatory or inhibitory effect on, for example, separase expression, separase phosphorylation or separase activity, or have a stimulatory or inhibitory effect on, for example, the expression or activity of separase substrate (e.g., cleavage of the chromosomal cohesin^{SCC1}).

As used herein, the term “small organic molecule” refers to an organic molecule, either naturally occurring or synthetic, that has a molecular weight of more than about 25 daltons and less than about 3000 daltons, preferably less than about 2500 daltons, more preferably less than about 2000 daltons, preferably between about 100 to about 1000 daltons, more preferably between about 200 to about 500 daltons.

In one embodiment, the invention provides assays for screening candidate or test compounds which are substrates of separase or a separase polypeptide or biologically active portion thereof. In another embodiment, the invention provides assays for screening candidate or test compounds which bind to or modulate the activity of separase or a separase polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412), or on beads (Lam (1991) Nature 354:82), chips (Fodor (1993) Nature 364:555), bacteria (Ladner U.S. Pat. No. 5,223,409), spores (Ladner U.S. Pat. No. 5,223,409), plasmids (Cull et al. (1992) Proc. Natl. Acad. Sci. USA 89:1865) or on phage (Scott and Smith (1990) Science 249:386); (Devlin (1990) Science 249:404); (Cwirla et al. (1990) Proc. Natl. Acad. Sci. USA 87:6378); (Felici (1991) J. Mol. Biol. 222:301); (Ladner supra).

Examples of methods for introducing a molecular library of randomized nucleic acids into a population of cells can be found in the art, for example in U.S. Pat. No. 6,365,344, incorporated herein in its entirety by reference. A molecular library of randomized nucleic acids can provide for the direct selection of candidate or test compounds with desired phenotypic effects. The general method can involve, for instance, expressing a molecular library of randomized nucleic acids in a plurality of cells, each of the nucleic acids comprising a different nucleotide sequence, screening for a cell of exhibiting a changed physiology in response to the presence in the cell of a candidate or test compound, and detecting and isolating the cell and/or candidate or test compound.

In one embodiment, the introduced nucleic acids are randomized and expressed in the cells as a library of isolated randomized expression products, which may be nucleic acids, such as mRNA, antisense RNA, siRNA, ribozyme components, etc., or peptides (e.g., cyclic peptides). The library should provide a sufficiently structurally diverse population of randomized expression products to effect a probabilistically sufficient range of cellular responses to provide one or more cells exhibiting a desired response. Generally at least 10⁶, preferably at least 10⁷ more preferably at least 10⁸ and most preferably at least 10⁹ different expression products are simultaneously analyzed in the subject methods. Preferred methods maximize library size and diversity.

The introduced nucleic acids and resultant expression products are randomized, meaning that each nucleic acid and peptide consists of essentially random nucleotides and amino acids, respectively. The library may be fully random or biased, e.g. in nucleotide/residue frequency generally or per position. In other embodiments, the nucleotides or residues are randomized within a defined class, e.g. of hydrophobic amino acids, of purines, etc. In any event, where the ultimate expression product is a nucleic acid, at least 10, preferably at least 12, more preferably at least 15, most preferably at least 21 nucleotide positions need to be randomized; more if the randomization is less than perfect. Similarly, at least 5, preferably at least 6, more preferably at least 7 amino acid positions need to be randomized; again, more if the randomization is less than perfect.

Functional and structural isolation of the randomized expression products may be accomplished by providing free (not covalently coupled) expression product, though in some situations, the expression product may be coupled to a functional group or fusion partner, preferably a heterologous (to the host cell) or synthetic (not native to any cell) functional group or fusion partner. Exemplary groups or partners include, but are not limited to, signal sequences capable of constitutively localizing the expression product to a predetermined subcellular locale such as the Golgi, endoplasmic reticulum, nucleoli, nucleus, nuclear membrane, mitochondria, chloroplast, secretory vesicles, lysosome, and the like; binding sequences capable of binding the expression product to a predetermined protein while retaining bioactivity of the expression product; sequences signaling selective degradation, of itself or co-bound proteins; and secretory and membrane-anchoring signals.

It may also be desirable to provide a partner which conformationally restricts the randomized expression product to more specifically define the number of structural conformations available to the cell. For example, such a partner may be a synthetic presentation structure: an artificial polypeptide capable of intracellularly presenting a randomized peptide as a conformation-restricted domain. Generally such presentation structures comprise a first portion joined to the N-terminal end of the randomized peptide, and a second portion joined to the C-terminal end of the peptide. Preferred presentation structures maximize accessibility to the peptide by presenting it on an exterior loop, for example of coiled-coils, (Myszka, D. G., and Chaiken, I. M. Design and characterization of an intramolecular antiparallel coiled coil peptide. Biochemistry. 1994. 33:2362-2372). To increase the functional isolation of the randomized expression product, the presentation structures are selected or designed to have minimal biologically active as expressed in the target cell. In addition, the presentation structures may be modified, randomized, and/or matured to alter the presentation orientation of the randomized expression product. For example, determinants at the base of the loop may be modified to slightly modify the internal loop peptide tertiary structure, while maintaining the absolute amino acid identity. Other presentation structures include zinc-finger domains, loops on beta-sheet turns and coiled-coil stem structures in which non-critical residues are randomized; loop structures held together by cysteine bridges, cyclic peptides, etc.

In another embodiment, the present invention provides cyclic peptides for use in the libraries described herein. As used herein, the term “cyclic peptide” refers to a peptide configured in a loop. Cyclic peptides can be produced by generating a nucleotide sequence encoding a peptide to be cyclized flanked on one end with a nucleotide sequence encoding the carboxy-terminal portion of a split (or trans) intein (C-intein or I_{C}) and on the other end with a nucleotide sequence encoding the amino-terminal portion of a split intein (N-intein or I_{N}). Expression of the construct within a host system, such as bacteria or eukaryotic cells described herein, results in the production of a fusion protein. The two split intein compounds (i.e., I_{C} and I_{N}) of the fusion protein then assemble to form an active enzyme that splices the amino and carboxy termini together to generate a backbone cyclic peptide. Cyclic polypeptides can be generated using a variety of inteins. Methods of generating cyclic proteins can be found in the art, for example, in WO 00/36093 and WO 01/57183, incorporated herein by reference in their entirety.

As used herein, the term “intein” refers to a naturally-occurring or artificially-constructed polypeptide embedded within a precursor protein that can catalyze a splicing reaction during post-translation processing of the protein.

In one embodiment, an assay is a cell-based assay in which a cell which expresses a separase protein or biologically active portion thereof is contacted with a test compound and the ability of the test compound to modulate separase activity, e.g., cleavage of cohesin^{SCC1} and/or separase, is determined. Determining the ability of the test compound to modulate separase activity can be accomplished by monitoring, for example, the phosphorylation of separase or the cleavage of separase target molecules. Determining the ability of the test compound to modulate the ability of separase to bind to a substrate can be accomplished, for example, by coupling the separase substrate with a radioisotope or enzymatic label such that binding of the separase substrate to separase can be determined by detecting the labeled separase substrate in a complex. For example, compounds (e.g., separase substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a separase substrate) to interact with separase without the labeling of any of the interactants. For example, a microphysiometer can be used to detect the interaction of a compound with separase without the labeling of either the compound or separase (McConnell, H. M. et al. (1992) Science 257:1906). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and separase.

In another embodiment, an assay is a cell-based assay comprising contacting a cell expressing a separase target molecule (e.g., cohesin^{SCC1}) with a test compound and determining the ability of the test compound to modulate (e.g. stimulate or inhibit, e.g., by cleavage) the activity of the separase target molecule. Determining the ability of the test compound to modulate the activity of a separase target molecule can be accomplished, for example, by determining the ability of the separase protein to bind to or interact with the separase target molecule, e.g., a cohesion^{SCC1} or a fragment thereof.

Determining the ability of separase or a biologically active fragment thereof, to bind to or interact with a separase target molecule can be accomplished by one of the methods described above for determining direct binding. In a preferred embodiment, determining the ability of separase to bind to or interact with a separase target molecule can be accomplished by determining the activity of the target molecule. For example, the activity of the target molecule can be determined by detecting induction of a cellular second messenger of the target, detecting catalytic/enzymatic activity of the target an appropriate substrate, detecting the induction of a reporter gene (comprising a target-responsive regulatory element operatively linked to a nucleic acid encoding a detectable marker, e.g., luciferase), or detecting a target-regulated cellular response (e.g., sister chromatid separation).

In yet another embodiment, an assay of the present invention is a cell-free assay in which separase or biologically active portion thereof is contacted with a test compound and the ability of the test compound to bind to separase or a biologically active portion of separase is determined. Preferred biologically active portions of separase to be used in assays of the present invention include phosphorylation sites (e.g., S1073, S1126, S1305, T1346, S1501, S1508, S1545 and S1552 of SEQ ID NO: 3); catalytic amino acids (e.g., cysteine 2029 of SEQ ID NO:3); and autocatalytic cleavage sites (e.g., R1486, R1506, and R1535 of SEQ ID NO:3). Binding of the test compound to separase can be determined either directly or indirectly as described above. In a preferred embodiment, the assay includes contacting separase or biologically active portion of separase with a known compound which binds separase to form an assay mixture, contacting the assay mixture with a test compound, and determining the ability of the test compound to interact with separase, wherein determining the ability of the test compound to interact with separase comprises determining the ability of the test compound to preferentially bind to separase or a biologically active portion of separase as compared to the known compound.

In another embodiment, the assay is a cell-free assay in which separase or a biologically active portion of separase is contacted with a test compound and the ability of the test compound to modulate (e.g., stimulate or inhibit) the activity of separase or a biologically active portion of separase is determined. Determining the ability of the test compound to modulate the activity of separase can be accomplished, for example, by determining the ability of separase to bind to a separase target molecule by one of the methods described above for determining direct binding. Determining the ability of separase to bind to a separase target molecule can also be accomplished using a technology such as real-time Biomolecular Interaction Analysis (BIA) (Sjolander, S. and Urbaniczky, C. (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of separase can be accomplished by determining the ability of separase to further modulate the activity of a downstream effector of a separase target molecule. For example, the activity of the effector molecule on an appropriate target can be determined or the binding of the effector to an appropriate target can be determined as previously described.

In yet another embodiment, the cell-free assay involves contacting separase or a biologically active portion of separase with a known compound which binds separase to form an assay mixture, contacting the assay mixture with a test compound, and determining the ability of the test compound to interact with separase, wherein determining the ability of the test compound to interact with separase comprises determining the ability of separase to preferentially bind to or modulate the activity of a separase target molecule (e.g., separase phosphorylation, securin cleavage, separase cleavage and the like).

In more than one embodiment of the above assay methods of the present invention, it may be desirable to immobilize either separase or its target molecule to facilitate separation of complexed from uncomplexed forms of one or both of the proteins, as well as to accommodate automation of the assay. Binding of a test compound to separase, or interaction of separase with a target molecule in the presence and absence of a candidate compound, can be accomplished in any vessel suitable for containing the reactants. Examples of such vessels include microtitre plates, test tubes, and microfuge tubes. In one embodiment, a fusion protein can be provided which adds a domain that allows one or both of the proteins to be bound to a matrix. For example, glutathione-S-transferase/separase fusion proteins or glutathione-S-transferase/target fusion proteins can be adsorbed onto glutathione sepharose beads (Sigma, St. Louis, Mo.) or glulathione-derivatized microtitre plates, which are then combined with the test compound or the test compound and either the non-adsorbed target protein or separase, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of separase binding or activity determined using standard techniques.

Other techniques for immobilizing proteins on matrices can also be used in the screening assays of the invention. For example, either separase or a separase target molecule can be immobilized utilizing conjugation of biotin and avidin or streptavidin. Biotinylated separase or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce). Alternatively, antibodies reactive with separase or target molecules that do not interfere with binding of separase to its target molecule can be derivatized to the wells of the plate, and unbound target or separase trapped in the wells by antibody conjugation. Methods for detecting such complexes, in addition to those described above for the GST-immobilized complexes, include immunodetection of complexes using antibodies reactive with separase or separase target molecule, as well as enzyme-linked assays which rely on detecting an enzymatic activity associated with separase or separase target molecule.

In another embodiment, modulators of separase expression and/or separase phosphorylation are identified in a method wherein a cell is contacted with a candidate compound and the expression of separase protein, separase mRNA, and/or the phosphorylation of separase (e.g., phosphorylation at S1126 and/or T1346 of SEQ ID NO:3) in the cell is determined. The level of separase protein, separase mRNA, and/or phosphorylated separase in the presence of the candidate compound is compared to the level of separase protein, separase mRNA, and/or phosphorylated separase in the absence of the candidate compound. The candidate compound can then be identified as a modulator of separase protein expression, separase mRNA expression, and/or separase phosphorylation based on this comparison. For example, when expression of separase protein, separase mRNA, and/or phosphorylated separase is greater (statistically significantly greater) in the presence of the candidate compound than in its absence, the candidate compound is identified as a stimulator of separase protein expression, separase mRNA expression, and/or separase phosphorylation, respectively. Alternatively, when expression of separase protein, separase mRNA, and/or phosphorylated separase is less (statistically significantly less) in the presence of the candidate compound than in its absence, the candidate compound is identified as an inhibitor of separase protein expression, separase mRNA expression, and/or separase phosphorylation, respectively. The level of separase mRNA or protein expression and separase phosphorylation in the cells can be determined by methods described herein for detecting separase mRNA or protein and separase phosphorylation.

In yet another aspect of the invention, separase can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO 94/10300), to identify other proteins, which bind to or interact with separase (“separase-binding proteins” or “separase-bp”) and are involved in separase activity (e.g., cohesion^{SCC1} cleavage, separase cleavage, and/or sister chromatid separation). Such separase-binding proteins are also likely to be involved in the propagation of signals by separase or separase targets as, for example, downstream elements of a separase-mediated signaling pathway. Alternatively, such separase-binding proteins are likely to be separase inhibitors (such as securin).

The two-hybrid system is based on the modular nature of most transcription factors, which consist of separable DNA-binding and activation domains. Briefly, the assay utilizes two different DNA constructs. In one construct, the gene that codes for separase is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a separase-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with separase.

In another embodiment, an assay is an animal model based assay comprising contacting a an animal with a test compound and determining the ability of the test compound to alter separase expression and/or separase phosphorylation. Preferably, the animal is an animal model of sister chromatid separation such as securin knock-out mice. Preferred animals include but are not limited to mammals such as non-human primates, rabbits, rats, mice, and the like.

This invention further pertains to novel agents identified by the above-described screening assays. Accordingly, it is within the scope of this invention to further use an agent identified as described herein in an appropriate animal model as described herein. For example, an agent identified as described herein (e.g., a separase modulating agent) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments of disorders associated with aberrant chromosome separation (e.g., aberrant sister chromatid separation) such as aneuploid-related disorders such as cancer, and disorders causing congential defects such as Down's syndrome and spontaneous fetal abortion, as described herein.

Recombinant Expression Vectors and Host Cells

Another aspect of the invention pertains to vectors, preferably expression vectors, containing a nucleic acid encoding a separase protein (or a portion thereof). As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., separase proteins, mutant forms of separase proteins, fusion proteins, and the like).

The recombinant expression vectors of the invention can be designed for expression of separase in prokaryotic or eukaryotic cells. For example, separase or separase fragments can be expressed in bacterial cells such as E. coli, insect cells (using baculovirus expression vectors), yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of polypeptides in prokaryotes is most often carried out in E. coli with vectors containing constitutive or inducible promoters directing the expression of either fusion or non-fusion proteins. Fusion vectors add a number of amino acids to a polypeptide encoded therein, usually to the amino terminus of the recombinant polypeptide. Such fusion vectors typically serve three purposes: 1) to increase expression of recombinant polypeptide; 2) to increase the solubility of the recombinant polypeptide; and 3) to aid in the purification of the recombinant polypeptide by acting as a ligand in affinity purification. Often, in fusion expression vectors, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Purified fusion polypeptide can be utilized in translation initiation activity assays, or to generate antibodies specific for phosphorylated separase, for example.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111). Such alteration of nucleic acid sequences of the invention can be carried out by standard DNA synthesis techniques.

In another embodiment, the separase expression vector is a yeast expression vector. Examples of vectors for expression in yeast S. cerevisiae include pYepSec1 (Baldari et al., (1987) Embo J. 6:229), pMFa (Kurjan and Herskowitz, (1982) Cell 30:933), pJRY88 (Schultz et al., (1987) Gene 54:113), pYES2 (Invitrogen Corporation, San Diego, Calif.), and picZ (InVitrogen Corp, San Diego, Calif.).

Alternatively, separase polypeptides can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156) and the pVL series (Lucklow and Summers (1989) Virology 170:31).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187). When used in mammalian cells, the expression vector's control functions are often provided by viral regulatory elements. For example, commonly used promoters are derived from polyoma, adenovirus 2, cytomegalovirus and Simian virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268), lymphoid-specific promoters (Calame and Eaton (1988) Adv. Immunol. 43:235), in particular promoters of T cell receptors (Winoto and Baltimore (1989) EMBO J. 8:729) and immunoglobulins (Banerji et al. (1983) Cell 33:729; Queen and Baltimore (1983) Cell 33:741), neuron-specific promoters (e.g., the neurofilament promoter; Byrne and Ruddle (1989) Proc. Natl. Acad. Sci. U.S.A. 86:5473), pancreas-specific promoters (Edlund et al. (1985) Science 230:912), and mammary gland-specific promoters (e.g., milk whey promoter; U.S. Pat. No. 4,873,316 and European Application Publication No. 264,166). Developmentally regulated promoters are also encompassed, for example the murine hox promoters (Kessel and Gruss (1990) Science 249:374) and the α-fetoprotein promoter (Campes and Tilghman (1989) Genes Dev. 3:537).

In one embodiment, the present invention provides a nucleic acid molecule which is antisense to a separase nucleic acid molecule. As used herein, the term “antisense” refers to a nucleic acid that interferes with the function of DNA and/or RNA and may result in suppression of expression of the RNA and/or DNA. The antisense nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire separase coding strand, or to only a portion thereof.

An antisense nucleic acid molecule can be delivered to a cell to express an exogenous nucleotide sequence, to inhibit, eliminate, augment, or alter expression of an endogenous nucleotide sequence, or to express a specific physiological characteristic not naturally associated with the cell. In a preferred embodiment, the antisense nucleic acid is an antisense RNA, an interfering double stranded RNA (“dsRNA”) or a short interfering RNA (“siRNA”).

As used herein, the term “siRNA” refers to double-stranded RNA that is less than 30 bases and preferably 21-25 bases in length. siRNA may be prepared by any method known in the art. For a review, see Nishikura (2001) Cell 16:415. In one embodiment, single-stranded, gene-specific sense and antisense RNA oligomers with overhanging 3′ deoxynucleotides are prepared and purified. For example, two oligomers, can be annealed by heating to 94° C. for 2 minutes, cooling to 90° C. for 1 minute, and then cooling to 20° C. at a rate of 1° C. per minute. The siRNA can then be injected into an animal or delivered into a desired cell type using methods of nucleic acid delivery described herein.

Another aspect of the invention pertains to host cells into which a recombinant expression vector of the invention has been introduced, containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, host cells can be bacterial cells such E. coli, insect cells, yeast, Xenopus cells, or mammalian cells (such as Chinese hamster ovary cells (CHO), African green monkey kidney cells (COS), or fetal human cells (293T)). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a detectable translation product or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a separase protein. Accordingly, the invention further provides methods for producing a separase protein using the host cells of the invention. In one embodiment, the method comprises culturing the host cell of invention (into which a recombinant expression vector encoding a detectable translation product has been introduced) in a suitable medium such that a detectable translation product is produced. In another embodiment, the method further comprises isolating a separase protein from the medium or the host cell.

The host cells of the invention can also be used to produce nonhuman transgenic animals. For example, in one embodiment, a host cell of the invention is a fertilized oocyte or an embryonic stem cell into which separase-coding sequences have been introduced. Such host cells can then be used to create non-human transgenic animals in which exogenous separase sequences have been introduced into their genome. Such animals are useful for studying the function and/or activity of separase and for identifying and/or evaluating modulators of separase activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a separase-encoding nucleic acid into the male pronuclei of a fertilized oocyte, e.g., by microinjection, retroviral infection, and allowing the oocyte to develop in a pseudopregnant female foster animal. The separase cDNA sequence of SEQ ID NO:2 can be introduced as a transgene into the genome of a non-human animal. Alternatively, a nonhuman homologue of a human separase gene, such as a mouse or rat separase gene, can be used as a transgene. Alternatively, a separase gene homologue, such as another separase family member, can be isolated based on hybridization to the separase cDNA sequences of SEQ ID NO:2 and used as a transgene. Intronic sequences and polyadenylation signals can also be included in the transgene to increase the efficiency of expression of the transgene. A tissue-specific regulatory sequence(s) can be operably linked to a detectable translation product transgene to direct expression of a detectable translation product to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al., and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a detectable translation product transgene in its genome and/or expression of detectable translation product mRNA in tissues or cells of the animals. A transgenic founder animal can then be used to breed additional animals carrying the transgene. Moreover, transgenic animals carrying a transgene encoding a detectable translation product can further be bred to other transgenic animals carrying other transgenes.

To create a homologous recombinant animal, a vector is prepared which contains at least a portion of a separase gene into which a deletion, addition or substitution has been introduced to thereby alter, e.g., functionally disrupt, the separase gene. The separase gene can be a human gene (e.g., the cDNA of SEQ ID NO:2), but more preferably, is a non-human homologue of a human separase gene. For example, a mouse separase gene can be used to construct a homologous recombination vector suitable for altering an endogenous separase gene in the mouse genome. In a preferred embodiment, the vector is designed such that, upon homologous recombination, the endogenous separase gene is functionally disrupted (i.e., no longer encodes a functional protein; also referred to as a “knock out” vector). Alternatively, the vector can be designed such that, upon homologous recombination, the endogenous separase gene is mutated or otherwise altered but still encodes functional protein (e.g., the upstream regulatory region can be altered to thereby alter the expression of the endogenous separase protein). In the homologous recombination vector, the altered portion of the separase gene is flanked at its 5′ and 3′ ends by additional nucleic acid sequence of the separase gene to allow for homologous recombination to occur between the exogenous separase gene carried by the vector and an endogenous separase gene in an embryonic stem cell. The additional flanking separase nucleic acid sequence is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced separase gene has homologously recombined with the endogenous separase gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-humans animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. U.S.A. 89:6232. Another example of a recombinase system is the FLP recombinase system of S. cerevisiae (O'Gorman et al. (1991) Science 251:1351). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810. In brief, a cell, e.g., a somatic cell, from the transgenic animal can be isolated and induced to exit the growth cycle and enter G₀ phase. Alternatively, a cell, e.g., an embryonic stem cell, from the inner cell mass of a developing embryo can be transformed with a preferred transgene. Alternatively, a cell, e.g., a somatic cell, from cell culture line can be transformed with a preferred transgene and induced to exit the growth cycle and enter G₀ phase. The cell can then be fused, e.g., through the use of electrical pulses, to an enucleated mammalian oocyte. The reconstructed oocyte is then cultured such that it develops to morula or blastocyst and then transferred to pseudopregnant female foster animal. The offspring borne of this female foster animal will be a clone of the animal from which the nuclear donor cell, e.g., the somatic cell, is isolated.

Diagnostic Assays

An exemplary method for detecting the presence or absence of separase protein, separase nucleic acid, or separase protein phosphorylation in a biological sample involves obtaining a biological sample from a test subject and contacting the biological sample with a compound or an agent capable of detecting separase protein (e.g., phosphorylated or unphosphorylated separase protein) or nucleic acid (e.g., mRNA, genomic DNA) that encodes separase protein such that the presence of separase protein or nucleic acid is detected in the biological sample. A preferred agent for detecting separase mRNA or genomic DNA is a labeled nucleic acid probe capable of hybridizing to separase mRNA or genomic DNA. The nucleic acid probe can be, for example, a full-length separase nucleic acid, such as the nucleic acid of SEQ ID NO:2, or a portion thereof, such as an oligonucleotide of at least 15, 30, 50, 100, 250, 500, 1000, 1500, 2000, 2500, 3000, 3500, 4000, 4500, 5000, 5500, or 6000 nucleotides in length and sufficient to specifically hybridize under stringent conditions to separase mRNA or genomic DNA. Other suitable probes for use in the diagnostic assays of the invention are described herein.

A preferred agent for detecting separase protein is an antibody capable of binding to separase protein, preferably an antibody with a detectable label. The antibody may bind only to phosphorylated separase protein, to unphosphorylated separase protein, or to either both phosphorylated and unphosphorylated separase protein. Antibodies can be polyclonal, or more preferably, monoclonal. An intact antibody, or a fragment thereof (e.g., Fab or F(ab′).sub.2) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect separase mRNA, protein, or genomic DNA in a biological sample in vitro as well as in vivo. For example, in vitro techniques for detection of separase mRNA include Northern hybridizations and in situ hybridizations. In vitro techniques for detection of separase protein include enzyme linked immunosorbent assays (ELISAs), Western blots, immunoprecipitations and immunofluorescence. In vitro techniques for detection of separase genomic DNA include Southern hybridizations. Furthermore, in vivo techniques for detection of separase protein include introducing into a subject a labeled anti-separase antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting separase protein, mRNA, or genomic DNA, or phosphorylated separase, such that the presence of separase protein, mRNA or genomic DNA, or phosphorylated separase is detected in the biological sample, and comparing the presence of separase protein, mRNA or genomic DNA, or phosphorylated separase in the control sample with the presence of separase protein, mRNA or genomic DNA, or phosphorylated separase, in the test sample.

The invention also encompasses kits for detecting the presence of separase in a biological sample. For example, the kit can comprise a labeled compound or agent capable of detecting separase protein or mRNA or phosphorylated separase in a biological sample; means for determining the amount of separase or phosphorylated separase in the sample; and means for comparing the amount of separase or phosphorylated separase in the sample with a standard. The compound or agent can be packaged in a suitable container. The kit can further comprise instructions for using the kit to detect separase protein or nucleic acid or phosphorylated separase.

Prognostic Assays

The diagnostic methods described herein can furthermore be utilized to identify subjects having or at risk of developing a disease or disorder associated with aberrant separase expression or activity (e.g., aberrant separase phosphorylation). For example, the assays described herein, such as the preceding diagnostic assays or the following assays, can be utilized to identify a subject having or at risk of developing a disorder associated with a misregulation in separase protein activity or nucleic acid expression, such as disorders associated with aberrant sister chromatid separation including aneuploid-related disorders such as cancer, and disorders causing congenital defects such as Down's syndrome and spontaneous fetal abortion.

Thus, the present invention provides a method for identifying a disease or disorder associated with aberrant separase expression or activity in which a test sample is obtained from a subject and separase protein (e.g., separase phosphorylation) or nucleic acid (e.g., mRNA or genomic DNA) is detected, wherein the presence of separase protein or nucleic acid is diagnostic for a subject having or at risk of developing a disease or disorder associated with aberrant separase expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant separase expression or activity. For example, such methods can be used to determine whether a subject can be effectively treated with an agent for cancer. Thus, the present invention provides methods for determining whether a subject can be effectively treated with an agent for a disorder associated with aberrant separase expression or activity in which a test sample is obtained and separase protein or nucleic acid expression or activity is detected (e.g., wherein the abundance of separase protein or nucleic acid expression or activity or separase phosphorylation is diagnostic for a subject that can be administered the agent to treat a disorder associated with aberrant separase expression or activity or aberrant separase phosphorylation).

The methods of the invention can also be used to detect genetic alterations in a separase gene, thereby determining if a subject with the altered gene is at risk for a disorder characterized by misregulation in separase protein activity or nucleic acid expression or separase phosphorylation, such as cancer. In preferred embodiments, the methods include detecting, in a sample of cells from the subject, the presence or absence of a genetic alteration characterized by at least one of: an alteration affecting the integrity of a gene encoding a separase protein; the misexpression of the separase gene; or the aberrant phosphorylation of the separase protein. For example, such genetic alterations can be detected by ascertaining the existence of at least one of: 1) a deletion of one or more nucleotides from a separase gene; 2) an addition of one or more nucleotides to a separase gene; 3) a substitution of one or more nucleotides of a separase gene, 4) a chromosomal rearrangement of a separase gene; 5) an alteration in the level of a messenger RNA transcript of a separase gene; 6) aberrant modification of a separase gene, such as of the methylation pattern of the genomic DNA; 7) the presence of a non-wild type splicing pattern of a messenger RNA transcript of a separase gene; 8) a non-wild type level of a separase protein; 9) allelic loss of a separase gene; and 10) inappropriate post-translational modification of a separase protein (e.g., inappropriate phosphorylation). As described herein, there are a large number of assays known in the art which can be used for detecting alterations in a separase gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077; and Nakazawa et al. (1994) Proc. Natl. Acad. Sci. USA 91:360), the latter of which can be particularly useful for detecting point mutations in the separase gene (see Abravaya et al. (1995) Nucleic Acids Res. 23:675). This method can include the steps of collecting a sample of cells from a subject, isolating nucleic acid (e.g., genomic, mRNA or both) from the cells of the sample, contacting the nucleic acid sample with one or more primers which specifically hybridize to a separase gene under conditions such that hybridization and amplification of the separase gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874), transcriptional amplification system (Kwoh, D. Y. et al., (1989) Proc. Natl. Acad. Sci. USA 86:1173), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a separase gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in separase can be identified by hybridizing a sample and control nucleic acids, e.g., DNA or RNA, to high density arrays containing hundreds or thousands of oligonucleotides probes (Cronin, M. T. et al. (1996) Human Mutation 7: 244; Kozal, M. J. et al. (1996) Nature Medicine 2:753). For example, genetic mutations in separase can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the separase gene and detect mutations by comparing the sequence of the sample separase with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147).

Other methods for detecting mutations in the separase gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type separase sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397; Saleeba et al. (1992) Methods Enzymol. 217:286. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in separase cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657). According to an exemplary embodiment, a probe based on a separase sequence, e.g., a wild-type separase sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in separase genes. For example, single strand conformation polymorphism (SSCP) may be used to detect differences in electrophoretic mobility between mutant and wild type nucleic acids (Orita et al. (1989) Proc. Natl. Acad. Sci. USA 86:2766, see also Cotton (1993) Mutat. Res. 285:125; and Hayashi (1992) Genet. Anal. Tech. Appl. 9:73). Single-stranded DNA fragments of sample and control separase nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nuc. Acids Res. 17:2437) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a separase gene and/or aberrant sister chromatid separation.

Furthermore, any cell type or tissue in which separase is expressed may be utilized in the prognostic assays described herein.

Monitoring of Effects During Clinical Trials

Monitoring the influence of agents (e.g., drugs) on the expression or activity of separase (e.g., the modulation of separase phosphorylation) can be applied not only in basic drug screening, but also in clinical trials. For example, the effectiveness of an agent determined by a screening assay as described herein to increase separase gene expression, or protein levels, to decrease phosphorylation, or upregulate separase activity, can be monitored in clinical trials of subjects exhibiting decreased separase gene expression, protein levels, downregulated separase activity, or increased separase phosphorylation. Alternatively, the effectiveness of an agent determined by a screening assay to decrease separase gene expression, or protein levels, to increase separase phosphorylation, or downregulate or increased separase phosphorylation activity, can be monitored in clinical trials of subjects exhibiting increased or increased separase phosphorylation gene expression, protein levels, or upregulated or increased separase phosphorylation activity, or decreased separase phosphorylation.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of a separase protein, mRNA, or genomic DNA or of the level of separase phosphorylation in the preadministration sample; (iii) obtaining one or more post-administration samples from the subject; (iv) detecting the level of expression or activity of the separase protein, mRNA, or genomic DNA or of the level of separase phosphorylation in the post-administration samples; (v) comparing the level of expression or activity of the separase protein, mRNA, or genomic DNA or of the level of separase phosphorylation in the pre-administration sample with the separase protein, mRNA, or genomic DNA or of the level of separase phosphorylation in the post administration sample or samples; and (vi) altering the administration of the agent to the subject accordingly. For example, increased administration of the agent may be desirable to increase the expression or activity of separase to higher levels than detected, i.e., to increase the effectiveness of the agent. Alternatively, decreased administration of the agent may be desirable to decrease expression or activity of separase to lower levels than detected, i.e. to decrease the effectiveness of the agent. According to such an embodiment, separase expression or activity may be used as an indicator of the effectiveness of an agent, even in the absence of an observable phenotypic response.

The following examples are provided for exemplification purposes only and are not intended to limit the scope of the invention which has been described in broad terms above.

### EXAMPLE 1

### Cloning of Human Separase

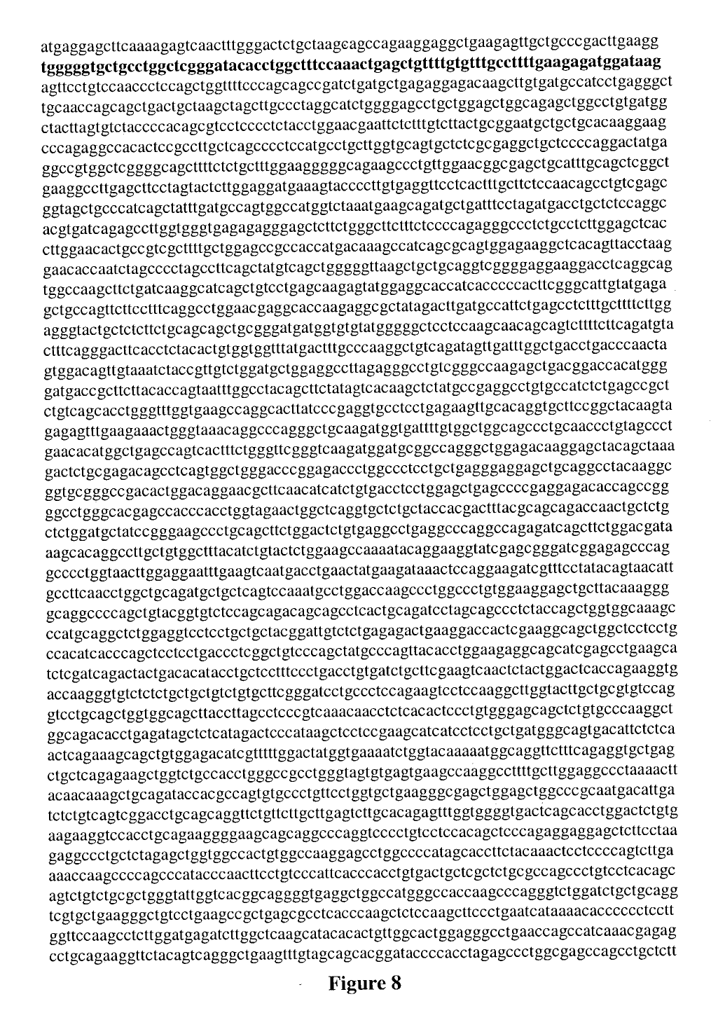

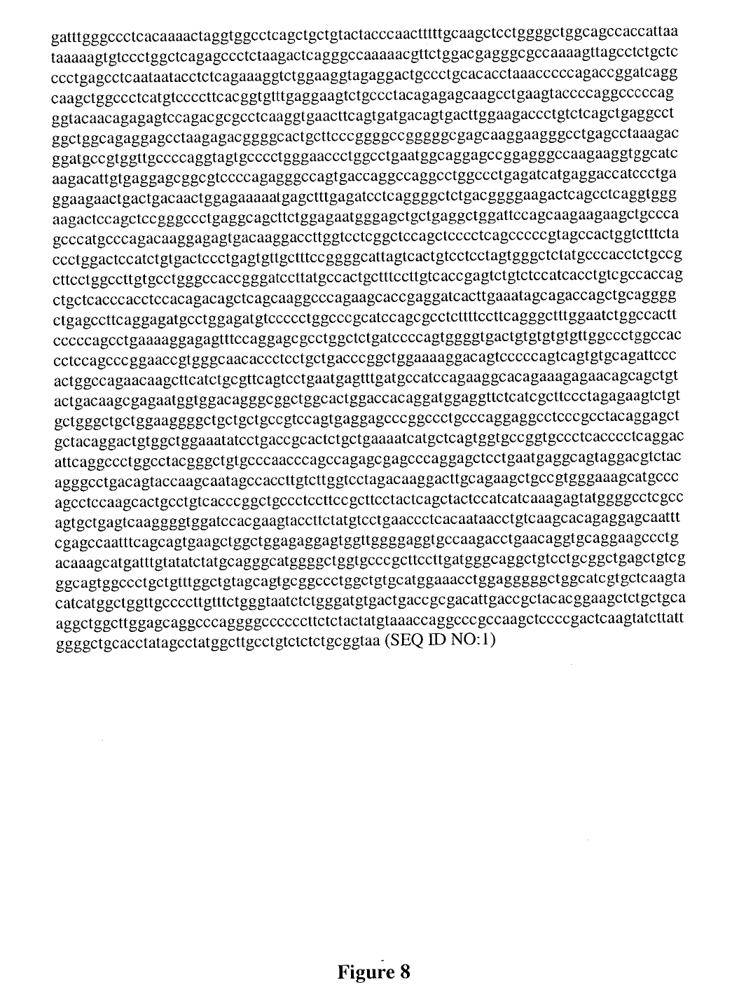

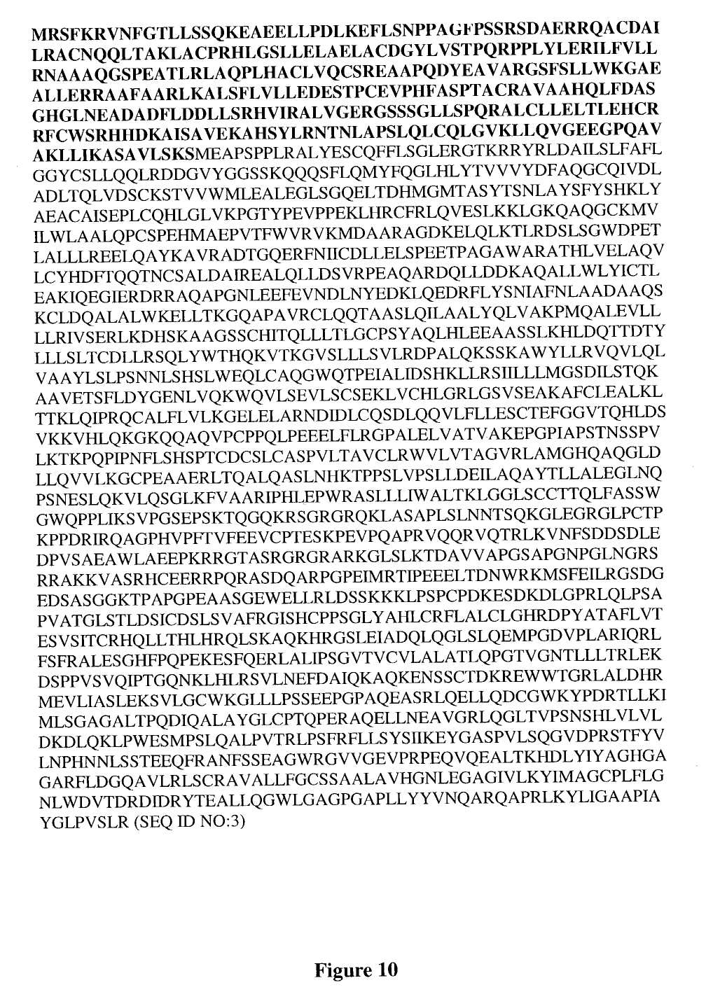

Full-length human separase was PCR-amplified from a human fetal thymus cDNA library (Clontech) using the following primers: 5′-ATGAGGAGCTTCAAAAGAGTCAACT TTGGGAC-3′ (SEQ ID NO:4) and 5′-TTACCGCAGAGAGACAGGCAAGCC-3′ (SEQ ID NO:5). The nucleotide sequence corresponding to the open reading frame of human separase containing a putative unspliced intron is set forth in FIG. 8 (SEQ ID NO:1). The nucleotide sequence corresponding to the open reading frame of human separase without the putative unspliced intron is set forth in FIG. 9 (SEQ ID NO:2). This open reading frame encodes the human separase protein (SEQ ID NO:3). The amino acid sequence of human separase is set forth in FIG. 10 (SEQ ID NO:3).

### EXAMPLE 2

### High CDC2 Activity Inhibits Anaphase and Sister Chromatid Separation in Xenopus Egg Extracts

The effect of CDC2 activity on sister chromatid separation and segregation was reinvestigated using Xenopus egg extracts (Shamu et al. (1992) J. Cell Biol. 117, 921). It was originally reported that non-degradable cyclinB1 lacking the N-terminal 90 amino acids including the destruction box (Δ90) prevents a mitotic exit and causes a stable arrest in late anaphase (Holloway et al. (1993) Cell 73:1393). Further analysis of this assay demonstrated sensitivity to the amount of Δ90 added.

His-tagged human cyclinB1Δ90 and recombinant securin were prepared as described (Kumagai et al. (1991) Cold Spring Harb. Symp. Quant. Biol. 56:585; Zou et al. (1999) Science 285, 418). The separase and hSCC1 antibodies were raised against an N-terminal peptide (RSFKRVNFGTLLSSQ (SEQ ID NO:6)) and a C-terminal peptide (EPYSDIIATPGPRFH (SEQ ID NO:7)) respectively (Genemed Synthesis). The anti-securin antibody was described elsewhere (Zou et al., 1999).

CSF extracts were prepared as described (Murray (1991) Methods Cell Biol. 36:581). For sister chromatid separation assays two sources of chromosomes were used, Xenopus sperm nuclei and isolated human metaphase chromosomes. Xenopus sperm nuclei were prepared as described by Philpott et al. ((1991) Cell 65:569). When they were used, the protocol developed by Murray and colleagues was followed (Holloway et al. (1993) Cell 73:1393) with the exception that Δ90 was usually added later, about 50 minutes after CSF. Re-isolation of Xenopus chromosomes was done according to Funabiki and Murray ((2000) Cell 102:411). When human chromosomes were used, the KCl concentration was lowered to 70 mM. Δ90 and isolated securin/separase (0.1 volumes) were added 20 minutes after addition of human chromosomes. After additional 20 minutes at 20° C., Ca²⁺ (0.6 mM) was added. Chromosomes were re-isolated from extracts 50 minutes thereafter. Analysis by immunofluorescence microscopy was performed as described (Wood et al. (1997) Cell 91:357). The percentage of separated chromosomes was calculated according to the following equation: % separation=(number of single chromatids/2)/[(number of single chromatids/2)+number of unseparated chromosomes].

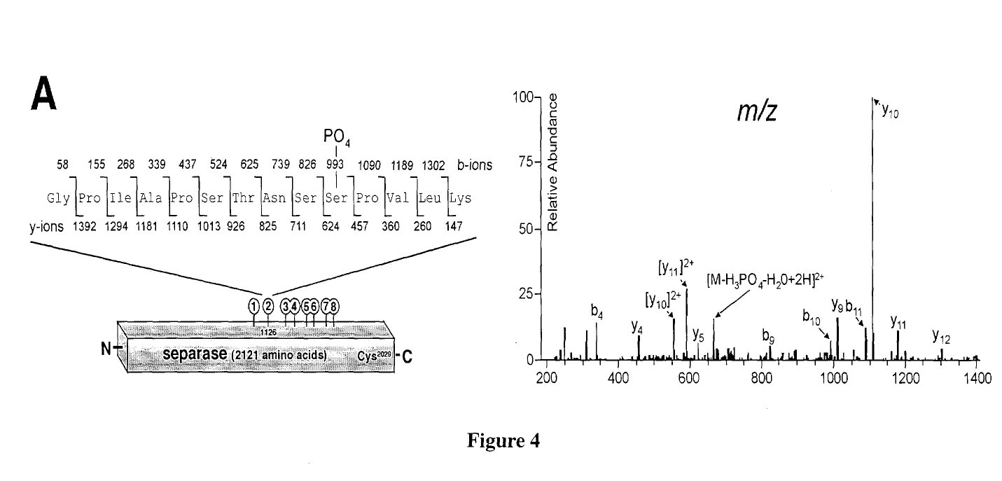

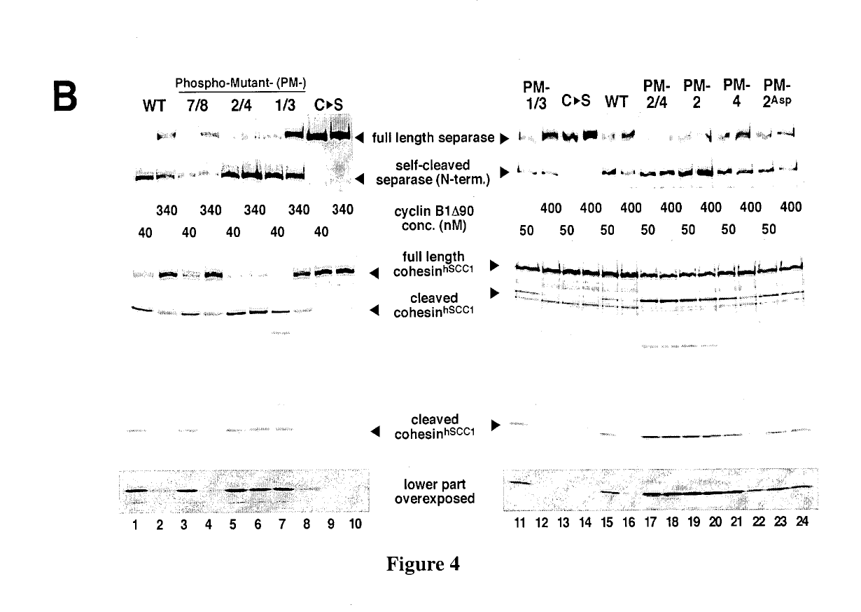

CSF-arrested Xenopus egg extracts supplemented with rhodamine-tubulin and Xenopus sperm nuclei were cycled through interphase and re-arrested at metaphase. Δ90 and roscovitine were added 25 and 10 minutes, respectively, before the addition of Ca²⁺. To evaluate anaphase occurrence, whole spindles and individual chromosomes were visualized by fluorescence microscopy (FIG. 4A). Spindle disassembly and chromosomes decondensation were used as readout for mitotic exit. Note that sister chromatid separation and segregation did actually occur at 0 and 20 nM Δ90 but were not evaluated because 50 minutes after calcium addition these extracts had long exited mitosis.

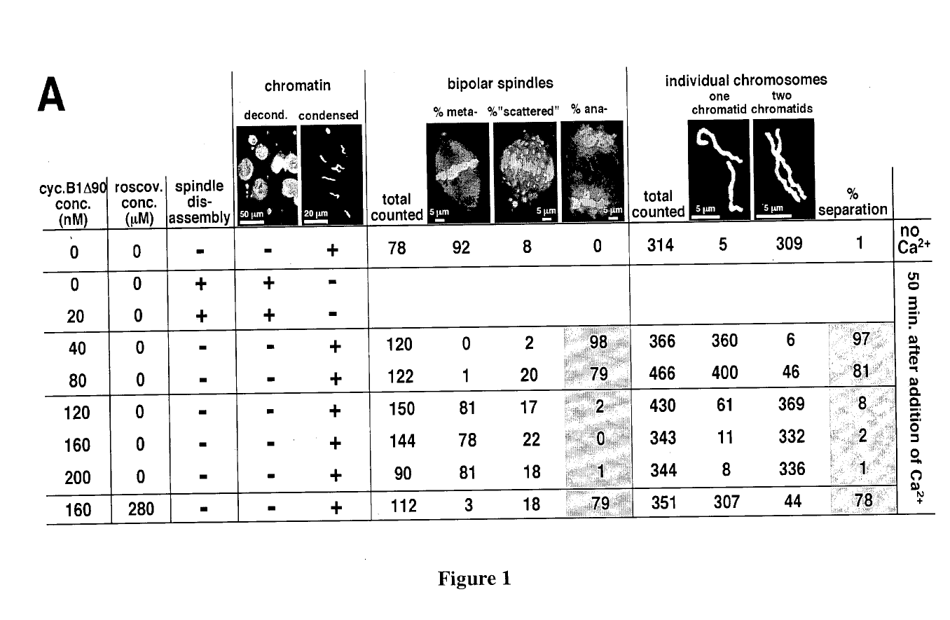

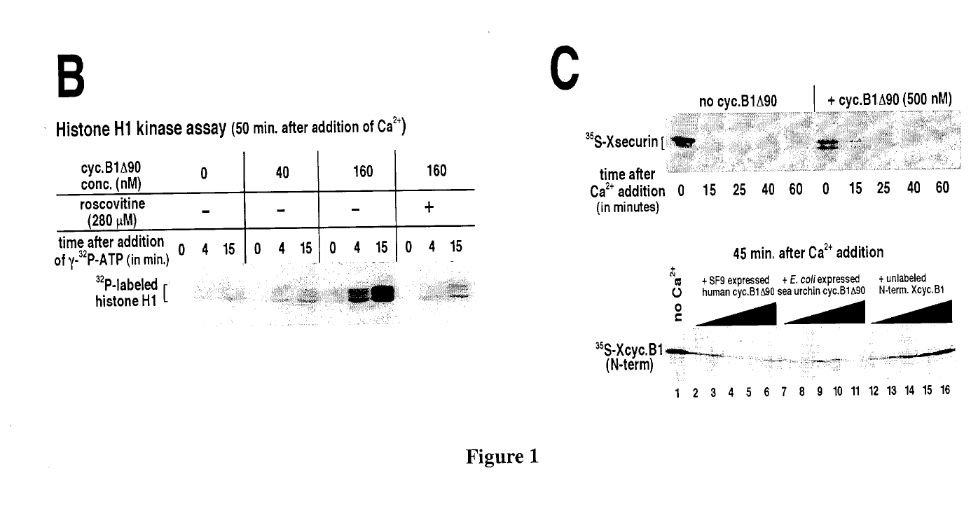

As reported by Holloway et al., supra, it was found that mitotic exit, as judged by spindle disassembly and chromosome decondensation, was blocked at a Δ90 concentration of at least 40 nM (FIG. 1A, columns 3 and 4). At this concentration, and up to 80 nM, anaphase occurred efficiently (FIG. 1A, rows 4 and 5). However, at a Δ90 concentration of 120 nM and above not only was mitotic exit prevented, but anaphase was also completely suppressed. Even 50 minutes after initiation of anaphase most spindles were still in a metaphase-like configuration (FIG. 1A, rows 6 to 8). This effect was not a peculiarity of the particular Δ90 preparation. In most of the experiments, the human Δ90 that was used was expressed in insect cells, but bacterially expressed sea urchin Δ90 caused the same inhibition phenotype (data not shown). Though demonstrating that a small (less than twofold) increase in Δ90 could change the terminal arrest phenotype, these observations did not clarify whether the spindles failed to move chromosomes towards the poles or whether the anaphase block was accompanied by a failure to dissolve sister chromatid cohesion. To address this issue, chromosomes were re-isolated from extracts 50 minutes after the initiation of anaphase and visualized by fluorescence microscopy at high magnification (Funabiki et al. (2000) Cell 102:411). This analysis revealed that at a Δ90 concentration of 40 to 80 nM almost all chromosomes displayed a one-chromatid configuration indicating that sister separation had taken place (FIG. 1A, rows 4 and 5). In contrast, at a Δ90 concentration of 120 nM and above most chromosomes displayed a butterfly-like shape characteristic for chromosomes composed of two unseparated chromatids (FIG. 1A, rows 6 to 8).

These data indicate that two different concentration ranges of non-degradable cyclinB1 cause two different effects. At the lower concentration range (40 to 80 nM) sister chromatid separation and segregation occur efficiently but mitotic exit is blocked; at the higher concentration range (above 120 nM) sister separation (and hence segregation) are inhibited as well. It is important to note that Δ90 completely blocks anaphase at a concentration only threefold higher than the minimal concentration necessary to prevent spindle disassembly and chromosome decondensation.

To determine whether Δ90 was poor APC substrate, the degradation of ³⁵S-labeled securin in extracts lacking Δ90 were compared to extracts containing Δ90 at a very high concentration (500 nM). The kinetics of degradation in both extracts were very similar (FIG. 1C, upper panel). Whether different Δ90 preparations could inhibit the degradation of an N-terminal fragment of cyclinB1, another well documented APC substrate (Glotzer et al. (1991) Nature 349:132; King et al. (1996b) Mol. Biol. Cell 7:1343), was also tested. Human or sea urchin Δ90 did not compete for the degradation of the ³⁵S-labeled fragment while an unlabeled fragment did so efficiently (FIG. 1C, lower panel). These data indicate that Δ90 is neither an APC substrate nor an APC inhibitor and therefore must inhibit anaphase by a mechanism other than by competitive inhibition of securin degradation.

Next, a specific CDC2 inhibitor, roscovitine, was used to assay whether Δ90 exerts its inhibitory effect by activating CDC2 or via an as yet unknown function. When anaphase was blocked in a high-Δ90 extract, addition of roscovitine rescued both events with high efficiency (FIG. 1A, row 9). At the same time roscovitine reduced the CDC2 activity to the level of a low-Δ90 extract, as determined by the histone H1 kinase assay, set forth below (FIG. 1B). This experiment demonstrated that non-degradable cyclinB1 acts by activating CDC2 and further that high CDC2 activity blocks sister chromatid separation in vitro.

For kinase assays, the securin/separase complex was isolated from unsynchronized, transfected cells and eluted with TEV protease without prior incubation in Xenopus extracts. CaMKII, CDC2/cyclinB1 and MAPK (ERK2) were from New England Biolabs and used as recommended. Reactions with polo and auroraA (a gift from E. A. Nigg) were carried out as described (Bischoff et al. (1998) EMBO J 17:3052; Descombes et al. (1998) EMBO J 17:1328). All kinase assays were done in the presence of 1 mM ATP.

### EXAMPLE 3

### High CDC2 Activity Inhibits Separase Activity in Xenopus Egg Extracts

To address whether high CDC2 activity blocked the activity of separase, an in vitro separase activity assay was developed. Plasmids coding for human securin and tagged human separase were transfected into 293T cells as follows.

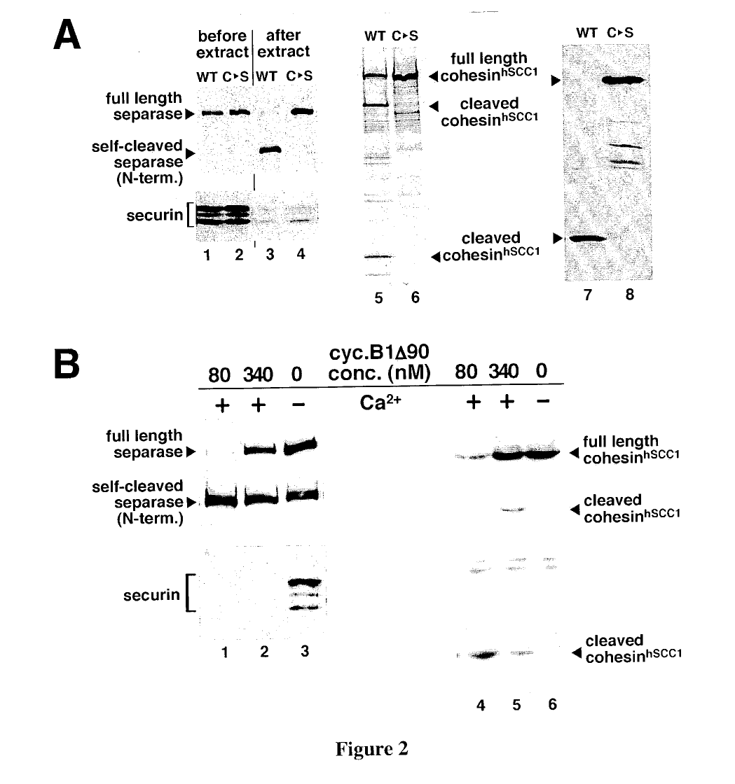

Human cohesin^{hSCC1} was isolated from the a human fetal thymus cDNA library using the following primers: 5′-ATGTTCTACGCACATTTTGTTCTCAG-3′ (SEQ ID NO:8) and 5′-TATAATATGGAACCTTGGTCCAGGTG-3′ (SEQ ID NO:9). Both separase and securin were subcloned into the multi-purpose expression vector pCS2 (various versions). The resulting plasmids were utilized for transfection into 293T cells and for in vitro expression in TNT™ reticulocyte lysate (Promega). For immunoprecipitation of separase, two types of N-terminal tags were fused to the amino terminus of separase, three HA tags (FIG. 2A) or two IgG binding domains of protein A followed by four TEV-protease cleavage sequences (ZZ-TEV₄-tag; FIGS. 2B, 4B, 5, 6B to E, and 7). Both versions of separase gave essentially the same results. Site directed mutagenesis was performed using either the QuickChange kit (Stratagene) or the GeneEditor system (Promega). All mutations were confirmed by DNA sequencing of manipulated regions.

To obtain securin/separase complexes, 293T cells were cotransfected with separase and securin expression plasmids using a calcium phosphate based method and subsequently synchronized as described (Fang et al. (1998) Mol. Cell 2:163). Two days after transfection, the nocodazole-arrested cells were lysed in 20 mM Tris/HCl pH 7.7, 100 mM NaCl, 1 mM NaF, 20 mM β-glycerophosphate, 5 mM MgCl₂, 0.1% Triton X100, 1 μM microcystin-LR. After ultra-centrifugation at 100,000 g, the supernatant was mixed with anti-HA agarose (3F10, Roche) or IgG-sepharose (Amersham), depending on the tag of separase. For a 10 cm dish of confluent, transfected cells (corresponding to roughly 100 μl cell pellet), 20 μl of beads were used. After rotation overnight at 4° C. the beads were washed twice with CSF-XB (Murray (1991) Methods Cell Biol. 36:581) and then incubated with various Xenopus egg extracts. To prepare low- or high-Δ90 extracts, CSF extracts were supplemented with various concentrations of Δ90. Fifteen minutes thereafter, Ca²⁺ was added (0.6 mM) and the extracts were further incubated for 15 minutes before adding them to the securin/separase beads. After 1 hour, the beads were washed twice with CSF-XB, once with 30 mM Hepes/KOH pH 7.7, 30% glycerol, 25 mM NaF, 25 mM KCl, 5 mM MgCl₂, 1.5 mM ATP, 1 mM EGTA, and eluted with HA peptide (1 mg/ml) or TEV-protease (2 mg/ml) in 20 to 50 μl. Two μl of separase were combined with 2 μl of in vitro translated ³⁵S-cohesin^{hSCC1} and incubated for 1 hour at 37° C. Alternatively, 2 μl of isolated metaphase chromosomes (8.7 μg DNA per μl) were used as a substrate.

After arrest in metaphase, the transfected cells were lysed and separase was isolated via its affinity tag. Separase was associated with its inhibitor securin and inactive at this stage (FIG. 2A, lane 1 and data not shown). When the complex was incubated in a low-Δ90 extract, securin was degraded (FIG. 2A, compare lanes 1 and 3). At the same time separase was cleaved resulting in two fragments migrating at 175 and 55 kDa (FIG. 2A, lane 3 and data not shown). This cleavage of separase is a characteristic of anaphase and occurs in vivo as well as in vitro (Waizenegger et al. (2000) Cell 103:399; Zou et al. (2002) FEBS Lett. 528:246). A mutant separase, in which the catalytic cysteine residue was replaced by a serine, was not cleaved under the same conditions (FIG. 2A, lane 4). As the active site lies far from where cleavage occurs, this result implies that the cleavage of separase is auto-catalyzed (Zou et al. (2002) FEBS Lett. 528:246). Self-cleavage of separase thus serves as a readout for separase activity. Re-isolation of securin-free separase from the Xenopus extract yielded a preparation that cleaved cohesin^{hSCC1} efficiently (FIG. 2A, lanes 5 to 8). This activity allowed the question of whether high-Δ90 extract had any impact on separase activity to be asked. As observed before, separase cleaved itself and cohesin effectively when treated with a low-Δ90 extract (FIG. 2B, lanes 1 and 4). Interestingly, both cleavage events were largely suppressed upon incubation in a high-Δ90 extract (FIG. 2B, lanes 2 and 5). Securin was degraded under both high and low Δ90 conditions (FIG. 2B, lanes 1 and 2), demonstrating once more that APC is active in a high-Δ90 extract. In contrast, securin was readily detected when APC is inhibited in a CSF extract (FIG. 2B, lane 3). Inhibition of APC is therefore not the reason for the inactivation of separase in a high-Δ90 extract. Taken together, these experiments demonstrate that in extracts with high CDC2 activity separase is kept inactive despite the absence of securin.

In some cases, a fraction of separase was already cleaved initially despite being fully inhibited, as measured by the activity assay. As the same degree of cleavage was detectable already in crude extracts, it was concluded that it occurred during synchronization of the cells. Cleavage to different extents was observed even for endogenous separase in untransfected cells (data not shown). The reason for these variations is not known but may indicate that self-cleaved separase can be re-inhibited (see below).

### EXAMPLE 4

### Phospho-Peptide Mapping of Separase

The inactivity of separase in extracts with high CDC2 activity indicated that separase might be negatively regulated by phosphorylation. To confirm negative regulation by phosphorylation, the endogenous securin/separase complex was purified from metaphase-arrested HeLaS3 cells and the phosphorylation sites were mapped by mass spectrometry.

The securin/separase complex was purified from high speed extracts of nocodazole-arrested HeLaS3 cells by ammonium sulfate precipitation and fractionation on SP-, S- and Q-ion exchange columns.

Metaphase chromosomes were isolated from HeLaS3 cells lysed in 5 mM Pipes/NaOH pH 7.2, 5 mM NaCl, 5 mM MgCl₂, 1 mM EGTA, 1% thiodiethylene glycol, complete protease inhibitor cocktail minus EDTA (Roche), 2.5 μM microcystin-LR, 1 μM okadaic acid, 1 mM ATP, 10 μg/ml cytochalasinB, and 0.2% digitonin by rate zonal centrifugation on a sucrose step gradient followed by isopycnic centrifugation in Percoll.

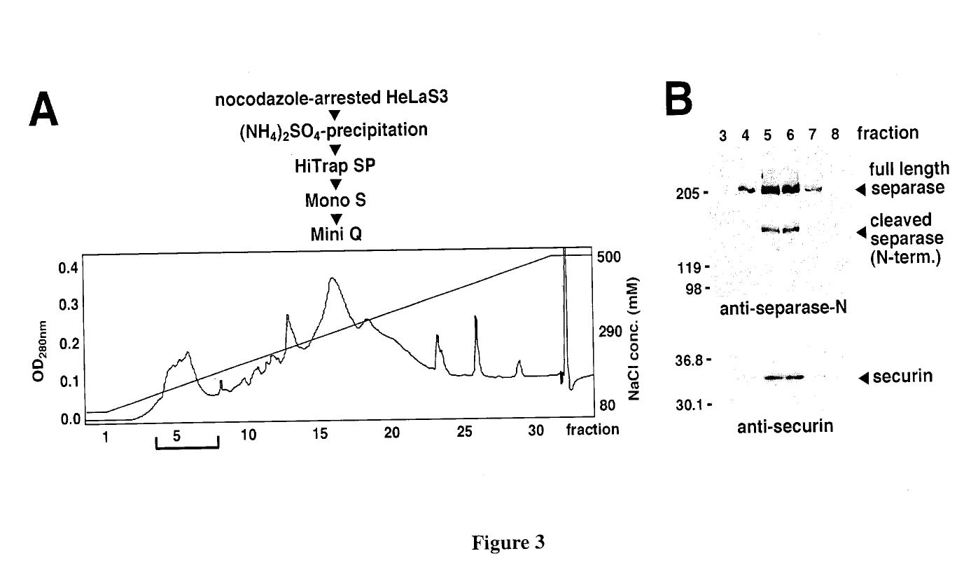

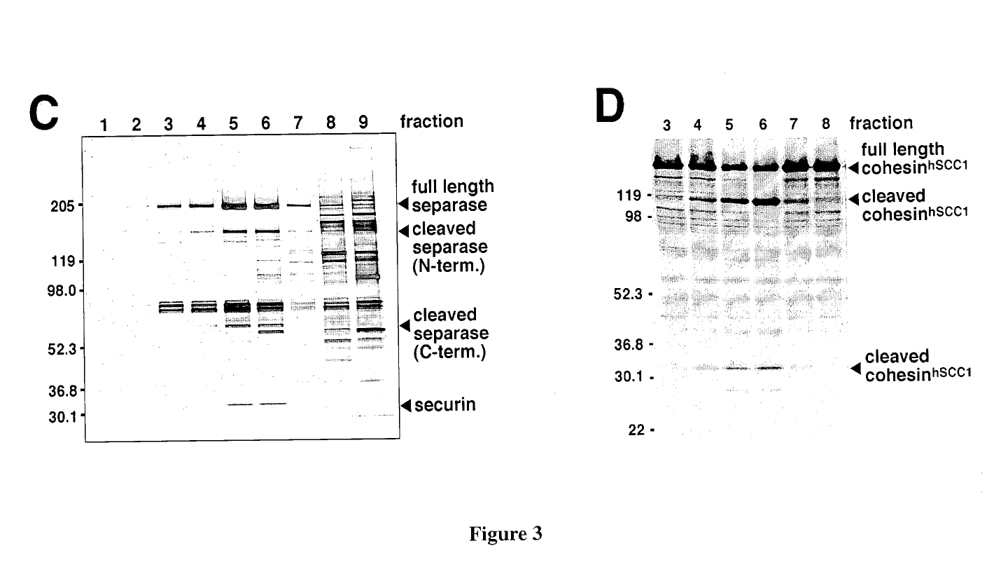

Western blots of the last purification step demonstrated that separase eluted together with securin (FIG. 3B). Separase and securin were among the few proteins that were detectable in this preparation by silver staining (FIG. 3C). No other major component seemed to co-fractionate with separase and securin. As expected, the securin/separase containing fractions cleaved ³⁵S-labeled cohesin^{hSCC1} only after securin was degraded by incubation with a low-Δ90 extract (FIG. 3D and data not shown).

After preparative SDS-Page and Coomassie staining, full-length separase and securin were cut from the gel, trypsin digested, and analyzed by LC-MS/MS. Phosphate-containing peptides were identified by a differential mass of +80 Da relative to the theoretical mass of unphosphorylated peptides. In this way, eight Ser/Thr-phosphorylation sites were identified for separase, all of which lie in the C-terminal half of the protein (FIG. 4A and data not shown). Phosphorylation site 2 turned out to be most important in regulating the activity of separase (set forth below). It was identified on two peptides of different length but spanning the same region (Glu1115 to Lys1130 and Gly1117 to Lys1130). In both cases, the analysis of the y- and b-ion fragmentation series revealed the presence of a phosphate group at Ser1126. As an example, the MS/MS-spectrum of the shorter phospho-peptide is shown in FIG. 4A. Ser165 of securin was also found to be phosphorylated (data not shown).

### EXAMPLE 5

### Separase is Regulated by Inhibitory Phosphorylation

All eight phosphorylation sites on separase were mutated to alanine, two at a time. The resulting phospho-mutants (PMs) were named according to the relative positions of the sites (FIG. 4A). Fragment ions in the spectrum represent mainly single-event preferential cleavage of the peptide bonds resulting in the sequence information recorded simultaneously from both the N- and C-termini (b- and y-type ions, respectively) of the peptide. This spectrum was computer-searched with the Sequest program (Eng et al., 1994) and was matched to a separase peptide with additional mass from a phosphate residue (sequence shown on the left side). With four potential sites of phosphorylation (three serines and one threonine), the correct assignment (Ser1126) was unambiguously determined based on the presence of ions derived by cleavage at the serine-serine peptide bond. This resulted in a b₉ (826 m/z) and y₅ [624 m/z, 544 (peptide)+80 (phosphate)].

They were expressed, purified, and tested for separase activity by the standard assay set forth above. Wild type separase cleaved itself and cohesin^{hSCC1} efficiently at low but not at high CDC2 activity (FIG. 4B, lanes 1, 2, 15, and 16). PM-1/3, -5/6, and -7/8 behaved like wild type separase (FIG. 4B, lanes 3, 4, 7, 8, 11, 12 and data not shown). Interestingly, PM-2/4 was no longer inhibited by incubation in extracts with high CDC2 activity; it cleaved itself and cohesin^{hSCC1} equally well under conditions of either low or high level of Δ90 (FIG. 4B, lanes 5, 6, 17, and 18). To elucidate the relative contributions of sites 2 and 4, single site mutants were generated. PM-2 behaved essentially like PM-2/4 and was still largely resistant to inhibition (FIG. 4B, lanes 19 and 20). PM-4 on the other hand was inactivated by high CDC2 activity albeit less so than wild type separase (FIG. 4B, lanes 21 and 22).

As controls, wild type separase (WT) and catalytic inactive separase (C [symbol] S) were also included. Each mutant was incubated in either low- (odd numbered lanes) or high-Δ90 extract (even numbered lanes) before analyzing separase self-cleavage by immuno-blot (top panels) and cohesin^{hSCC1} cleavage by autoradiography (middle and lower panels). Even when treated with low-Δ90 extracts, the separase activities of PM-2/4, PM-2, and—to a lesser extent—PM-4 are higher than that of wild type separase (compare lanes 17, 19, and 21 with lane 15) indicating the existence of a basal level of inhibitory phosphorylation in low-Δ90 extracts.

These results demonstrate that in vitro inhibition of separase in the absence of securin is due to phosphorylation of separase at one major site (Ser1126). This site resides roughly in the middle of the 233 kDa protease, far away from the catalytic residue (Cys2029). Mutation of Ser1126 to aspartate could not mimic phosphorylation. PM-2^{Asp} was not constitutively inhibited (FIG. 4B, compare lanes 15 and 23) but instead still largely resistant to inactivation by a high-Δ90 extract (FIG. 4B, lane 24).

### EXAMPLE 6

### A Single-Site Phospho-Mutant of Separase is Sufficient to Rescue Sister Chromatid Separation in a High-Δ90 Extract

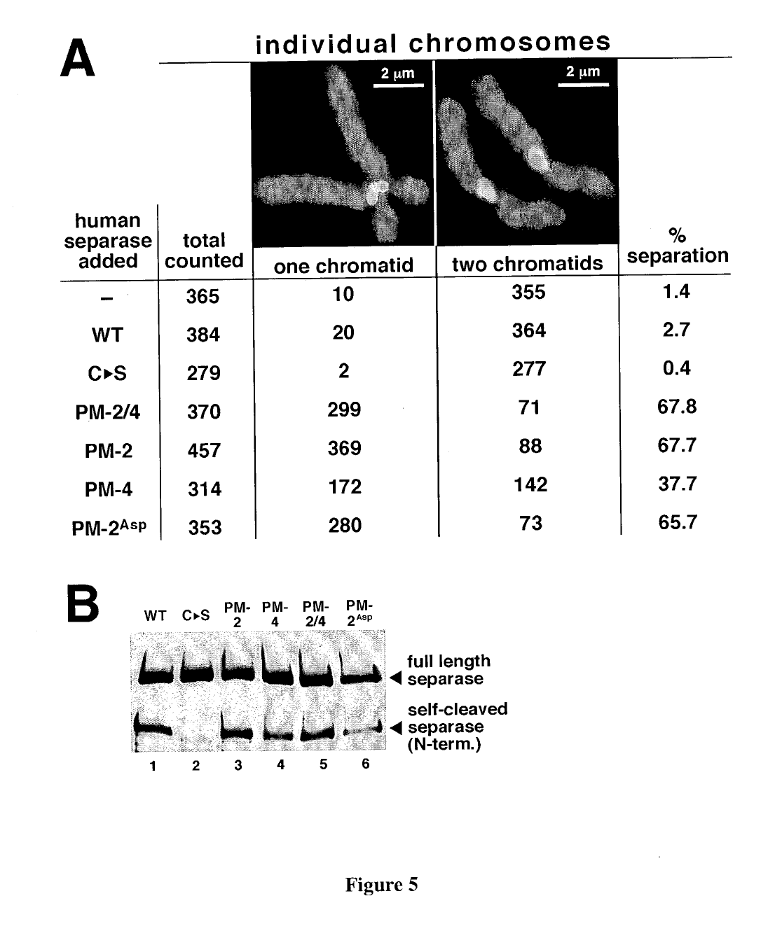

It was next determined whether the PM-2 mutant would override the inhibition not only of cohesin cleavage but also of sister chromatid separation in high-Δ90 extracts. Since the biochemical experiments utilized human separase, the existing assay was modified to examine the separation of human metaphase chromosomes in Xenopus extracts. The maximal degree of sister chromatid separation that was observed in this system under optimal conditions (low-Δ90 extract plus saturating level of PM-2/4) was about 70% (data not shown). In the presence of wild type separase (supplied as purified securin/separase complexes) and high concentrations of Δ90 (400 nm), only 2.7% of the chromosomes separated (FIG. 5A, row 2). Likewise, separation was negligible when catalytically inactive separase was added (0.4%) or when separase was omitted (1.4%; FIG. 5A, rows 1 and 3). In contrast, PM-2 (Ser 1126) led to maximal separation of sister chromatids (68%) under the same conditions (FIG. 5A, row 5). Similar results were obtained with PM-2/4 and PM-2^{Asp}; they caused 68% and 66% separation, respectively (FIG. 5A, rows 4 and 7). A Western blot for separase was performed to assure that similar amounts of the various separases had been used in the experiment (FIG. 5B).

These data indicate that preventing inhibitory phosphorylation of separase alone is sufficient to rescue sister chromatid separation in an extract with high CDC2 activity. The negative effect of a high-Δ90 extract on sister separation therefore seems to be mediated mostly, if not exclusively, by the inhibition of separase. Based on the above results the caveat that Cohesin^{hSCC1} might be rendered resistant to cleavage in an extract with high CDC2 activity can be excluded.

A second mutant, PM-4, caused some loss of cohesion, albeit less than PM-2 (38% versus 68%; FIG. 5A, rows 5 and 6). When the amount of added separase was reduced, the difference between PM-2 and -4 became more pronounced and resembled more closely the situation of the cohesin^{hSCC1} cleavage assay (FIG. 4B and data not shown). This indicates that phosphorylation site 4 (Thr1346) has a minor effect on separase activity while Ser1126 is the major regulatory site.

### EXAMPLE 7

### Separase Ser1126 is Quantitatively Phosphorylated in Metaphase Cells and Becomes Partly Dephosphorylated upon Anaphase Onset

Using a quantitative mass spectrometry technique, the phosphorylation of separase was quantified in synchronized cells. To this end, extracted peptides from in-gel digested separase were combined with a constant ratio of the synthetic, isotopically-labeled phosphorylated and unphosphorylated tryptic peptides Glu1115-Lys1130 (spanning Ser1126). These internal standards had the same sequence and therefore the same chemical properties as the native peptides but carried a heavy, ¹³C/¹⁵N-labeled leucine that increased their mass by seven Daltons relative to the native (light) peptides in the samples. Phosphorylated and unphosphorylated peptide levels could thereby be accurately determined by LC-MS/MS and compared between each sample.

For transfected 293T cells, it was found that 54 (+/−0.9)% of affinity-purified separase was phosphorylated at Ser1126 in nocodazole. 125 minutes after release from nocodazole, the level of phosphorylation dropped to 30 (+/−0.9)% (data not shown). Although affinity-purification of separase gave extremely clean peptide spectra, the transfection experiment had the disadvantage that it involved high overexpression of separase and that the synchronization of the cells was less efficient. It was therefore determined whether the phosphorylation of peptide Glu1115-Lys1130 could be measured under more physiological conditions and with as little manipulation as possible (i.e. with no purification of separase).

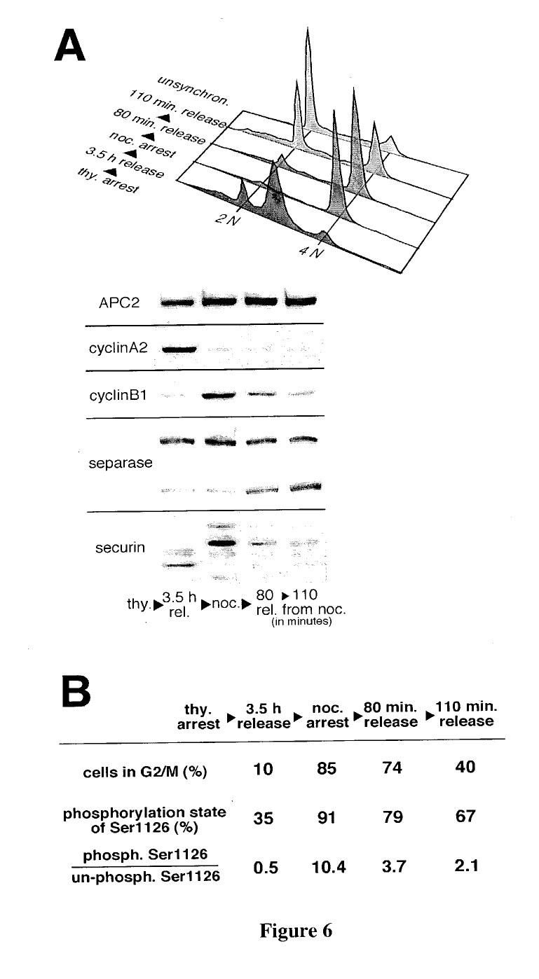

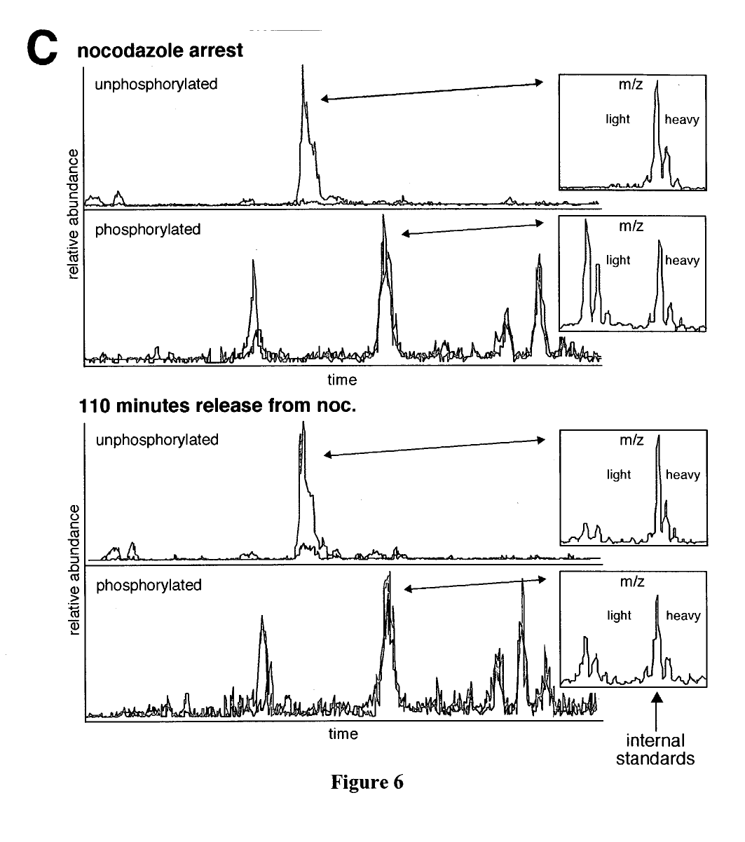

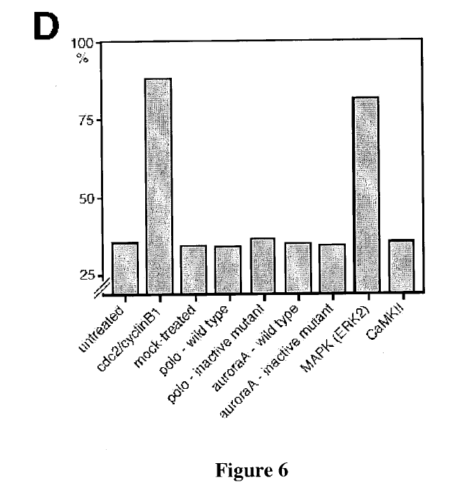

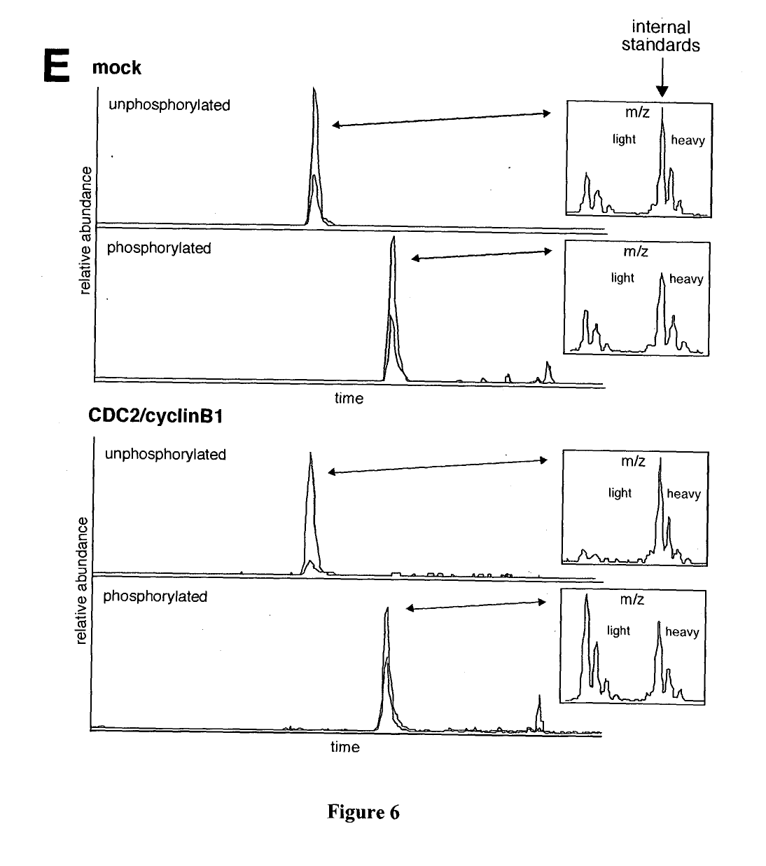

Crude high speed extracts from synchronized, un-transfected HeLaS3 cells were directly submitted to SDS-PAGE and the regions, where full-length separase and its N-terminal cleavage fragment migrated as judged by Western blotting, were cut from the gel. Because separase underwent self-cleavage upon release from nocodazole-arrest (FIG. 6A), both gel pieces of each time point were pooled and analyzed as described above. Remarkably, in using this technique, the Glu1115-Lys1130 peptides of endogenous separase could readily be detected from 0.4 mg total cell lysate. The analysis revealed that in metaphase 91% of Ser1126 was phosphorylated (FIGS. 6B and C). Immunoprecipitation of separase prior to the SDS-PAGE gave a very similar result (93% phosphorylation; data not shown), thereby confirming that the degree of phosphorylation was indeed accurately determined from crude extracts.

Given the fact that the arrest was not perfect (85% G2/M as determined by ModFit software) these results indicate that in metaphase, separase is quantitatively phosphorylated. Analysis of the other cell cycle states revealed that in S-phase, 35% of Ser1126 carried a phosphate residue. More importantly, 80 minutes after release from nocodazole only 79% of separase remained phosphorylated and this level dropped further to 67% at 110 minutes (FIGS. 6B and C). This change corresponds to a 5-fold decrease in the ratio of phosphorylated to unphosphorylated peptide. Considering that even at the 110 minutes time point 40% of the cells were still in mitosis as determined by FACS analysis (FIGS. 6A and B), these values represent approximately a 2-fold underestimation of the actual extent of dephosphorylation upon exit from mitosis. Overall, the relative change in the phosphorylation status of Ser1126 corresponded to the relative change of the cyclinB1 level (compare FIGS. 6A and B). In summary, these experiments demonstrated that separase becomes fully phosphorylated at its inhibitory site when cells are arrested in mitosis and that this phosphate group is removed from a considerable fraction of separase as cells undergo anaphase.

### EXAMPLE 8

### CDC2/cyclinB1 and MAP-Kinase Efficiently Phosphorylate Ser1126 In Vitro

Quantitative mass spectrometry was also used with isotopically labeled peptides to determine which kinase was able to phosphorylate separase in vitro at its inhibitory site. As a substrate, overexpressed securin/separase purified from transfected, unsynchronized 293T cells was used.

Coomassie stained securin and separase bands were digested in-gel (Shevchenko et al. (1996) Anal. Chem. 68:850). Extracted peptides were separated by nano-scale microcapillary high performance liquid chromatography (HPLC) as described (Gygi et al. (1999) Mol. Cell Biol. 19:1720). Eluting peptides were ionized by electrospray ionization and analyzed by an LCQ-DECA ion trap mass spectrometer (ThermoFinnigan). Peptide ions reaching a certain threshold were automatically selected for sequence analysis by tandem mass spectrometry. Peptide sequence was determined by data-searching against the non redundant human protein database using the Sequest algorithm (Eng et al. (1994) J. Am. Soc. Mass Spectrom. 5:1579).

Ser1126 was efficiently phosphorylated by both CDC2/cyclinB1 and MAPK (ERK2) but not at all by CaMKII (calmodulin-dependent kinase II), polo, or auroraA (FIGS. 6D and E). As controls, it was found that auroraA phosphorylated myelin basic protein and that CaMKII and polo underwent efficient autophosphorylation (data not shown).

### EXAMPLE 9

### Both Securin Binding and Phosphorylation Can Independently Inhibit Separase

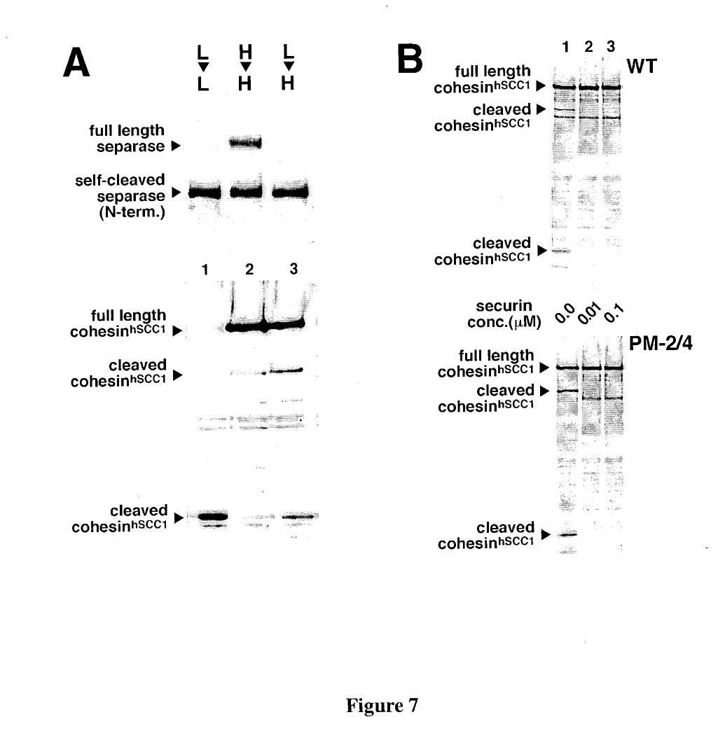

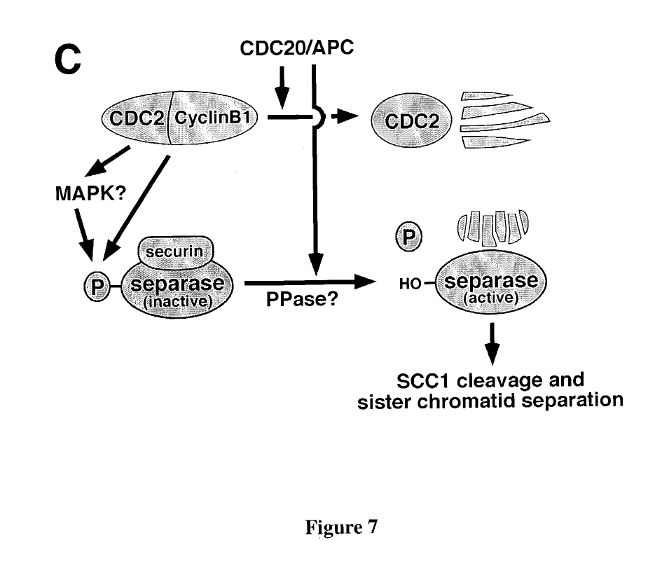

To determine whether securin can inhibit separase independent of the phosphorylation state of separase, and whether separase that has already been activated and has therefore cleaved itself be re-inhibited by either of the two inhibitory mechanisms, active separase was generated by treating a securin/separase complex with a low-Δ90 extract (L; FIG. 7) to degrade securin and dephosphorylate separase. The fact that separase had completely cleaved itself after this treatment demonstrated that it was indeed active at this state (data not shown). Active separase on beads was then incubated with either recombinant securin or a high-Δ90 extract and assayed for its ability to cleave cohesin^{hSCC1}. FIG. 7 illustrates that securin and a high-Δ90 extract (H; FIG. 7, lane 3) each caused re-inhibition of separase activity although the re-inhibition by phosphorylation was less complete (A, lane 3; B, lanes 2 and 3). Approximately a 2.5 fold molar excess of recombinant securin was sufficient to fully suppress cohesin^{hSCC1} cleavage (FIG. 6B). The respective control treatments left separase active (A, lane 1; B, lane 1). Likewise, separase did not become active when consecutively treated with high-Δ90 extract twice (FIG. 6A, lane 2).

The efficiency of inhibition of wild type and mutant separase by securin was also compared. When wild type separase and PM-2/4 were used in equal amounts, as judged by an anti-separase Western blot (FIG. 4B, lanes 15 and 17), both were inhibited at the same concentration of securin (FIG. 6B). Thus, wild type separase and the PM-2/4 mutant bind securin with similar affinities. This indicates that PM-2/4 is active in a high-Δ90 extract because it is no longer phosphorylated and not because it binds any residual securin, which might have escaped degradation, with much lower affinity.

### EXAMPLE 10

### A Revised Model for Sister Chromatid Separation

An extended model of sister chromatid separation in vertebrates based on the data presented herein is depicted in FIG. 7C. Before anaphase onset, separase is subject to a twofold inhibition: 1) The established inhibition of separase by association with the inhibitor securin; and 2) the novel inhibitory phosphorylation, which is due to the high CDC2/cyclinB1 activity at this stage of the cell cycle, described in the present invention. According to this model, securin degradation by its own is not sufficient to activate separase. Before sister chromatid separation can take place, the inhibitory phosphorylation has to be removed as well.

The data are consistent with APC causing destruction of a part of cyclinB1 thereby causing a drop in CDC2 activity. This would allow a putative, constitutively active phosphatase to gain the upper hand, which would result in dephosphorylation and activation of separase. Several observations support this explanation. In Xenopus extracts, histone H1 kinase activity drops to interphase level before anaphase becomes visible (Shamu et al. (1992) J. Cell Biol. 117, 921). Likewise, it has been reported in mammalian cells that cyclinB1 destruction commences about 25 minutes before anaphase onset. During the same period of time cyclinB1, which is localized to centrosomes and chromosomes, disappears (Clute et al. (1999) Nat. Cell Biol. 1:82). In this respect, it is noted that 1) separase also localizes to the centrosomes (H. Zou, O. Stemmann, and M. W. Kirschner, unpublished observation; D. Pellman, personal communication); and that 2) cohesin, which specifies the place of separase's ultimate action, is bound to chromosomes. Therefore, the early relocalization/degradation of cyclinB1 occurs at the right time and at the right place to support a model, in which a local drop of CDC2 activity causes a local activation of separase. Such a localized activation can explain why a complete dephosphorylation of separase was not detected upon release from nocodazole. Alternatively, the postulated phosphatase (see above) can be independently regulated and become active at the metaphase-anaphase transition. In this case it might dephosphorylate separase, despite a lack of cyclinB1 degradation.

### Equivalents

Those skilled in the art will recognize, or be able to ascertain using no more than routine experimentation, many equivalents to the specific embodiments and methods described herein. Such equivalents are intended to be encompassed by the scope of the following claims. All publications and patent applications cited above are incorporated by reference in their entirety for all purposes to the same extent as if each individual publication or patent application were specifically and individually indicated to be so incorporated by reference.

What is claimed:

1. A nucleic acid molecule which encodes a polypeptide having one or more separase activities, wherein the separase activities are selected from the group consisting of: cleaving cohesin^{hSCC1}, cleaving separase, and modulating sister chromatid separation.

2. A nucleic acid molecule which encodes a polypeptide comprising an amino acid sequence having at least about 60%, 70%, 80%, 90%, 95% or more sequence homology to an amino acid sequence of SEQ ID NO:3, wherein the polypeptide has one or more separase activities.

3. The polypeptide of claim 2, wherein the separase activities are selected from the group consisting of: cleaving cohesin^{hSCC1}, cleaving separase, and modulating sister chromatid separation.

4. A nucleic acid molecule comprising a nucleotide sequence having at least 60%, 70%, 80%, 90%, 95% or more sequence homology to a nucleotide sequence of SEQ ID NO:2, wherein the nucleotide sequence encodes a polypeptide having one or more separase activities.

5. The polypeptide of claim 4, wherein the separase activities are selected from the group consisting of: cleaving cohesin^{hSCC1}, cleaving separase, and modulating sister chromatid separation.

6. A nucleic acid molecule comprising a nucleotide sequence of SEQ ID NO:2 or analogs thereof.

7. A nucleic acid molecule consisting essentially of a nucleotide sequence of SEQ ID NO:2 or analogs thereof.

8. A nucleic acid molecule which encodes a polypeptide comprising an amino acid sequence of SEQ ID NO:3 or analogs thereof.

9. A nucleic acid molecule which encodes a polypeptide consisting essentially of an amino acid sequence of SEQ ID NO:3 or analogs thereof.

10. A nucleic acid molecule, selected from the group consisting of:
a) a nucleic acid molecule comprising a nucleotide sequence having at least about 98.9% sequence homology to a nucleotide sequence of SEQ ID NO:2, or a complement thereof;
b) a nucleic acid molecule which encodes a polypeptide comprising an amino acid sequence having at least about 85% sequence homology to an amino acid sequence of SEQ ID NO:3;
c) a nucleic acid molecule which encodes a fragment of a polypeptide, wherein the fragment comprises at least 10 contiguous amino acid residues of an amino terminal 325 amino acids of SEQ ID NO:3; and
d) a nucleic acid molecule which encodes a fragment of a polypeptide, wherein the fragment comprises at least 1,796 contiguous amino acids of SEQ ID NO:3.

11. A nucleic acid molecule, which hybridizes to a complement of the nucleic acid molecule of any one of claims 2, 4, 6, 8 or 10.

12. A nucleic acid molecule comprising a nucleotide sequence which is complementary to the nucleotide sequence of the nucleic acid molecule of any one of claims 2, 4, 6, 8 or 10.

13. A nucleic acid molecule of any one of claims 2, 4, 6, 8 or 10, further comprising a nucleotide sequence encoding a heterologous polypeptide.

14. A vector comprising the nucleic acid molecule of any one of claims 2, 4, 6, 8 or 10.

15. The vector of claim 14, which is an expression vector.

16. A host cell transfected with the expression vector of claim 15.

17. A method of producing a polypeptide comprising culturing the host cell of claim 16 in an appropriate culture medium to, thereby, produce the polypeptide.

18. A polypeptide having one or more separase activities, wherein the separase activities are selected from the group consisting of: cleaving cohesin^{hSCC1}, cleaving separase, and modulating sister chromatid separation.

19. A polypeptide comprising an amino acid sequence having at least about 60%, 70%, 80%, 90%, 95% or more sequence homology to an amino acid sequence of SEQ ID NO:3, wherein the polypeptide has one or more separase activities.

20. The polypeptide of claim 19, wherein the separase activities are selected from the group consisting of: cleaving cohesin^{hSCC1}, cleaving separase, and modulating sister chromatid separation.

21. A polypeptide comprising an amino acid sequence of SEQ ID NO:3 or analogs thereof.

22. A polypeptide consisting essentially of an amino acid sequence of SEQ ID NO:3 or analogs thereof.

23. A polypeptide selected from the group consisting of:
a) a polypeptide comprising a fragment of at least 10 contiguous amino acid residues of an amino terminal 325 amino acids of SEQ ID NO:3;
b) a polypeptide comprising a fragment of at least 1,796 contiguous amino acids of SEQ ID NO:3;
c) a polypeptide which is encoded by a nucleic acid molecule comprising a nucleotide sequence having at least about 98.9% sequence homology to a nucleic acid comprising a nucleotide sequence of SEQ ID NO:2; and
d) a polypeptide comprising an amino acid sequence having at least about 85% sequence homology to an amino acid sequence of SEQ ID NO:3.

24. The polypeptide of any one of claims 19, 21 or 23, further comprising heterologous amino acid sequences.

25. An antibody which selectively binds to a polypeptide of any one of claims 19, 21 or 23.

26. A method for detecting a polypeptide of any one of claims 19, 21 or 23 in a sample comprising the steps of:
a) contacting the sample with a compound which selectively binds to the polypeptide; and
b) determining whether the compound binds to the polypeptide in the sample to thereby detect the presence of the polypeptide in the sample.

27. The method of claim 26, wherein the compound which binds to the polypeptide is an antibody.

28. A kit comprising a compound which selectively binds to a polypeptide of any one of claims 19, 21 or 23 and instructions for use.

29. A method for detecting phosphorylation of the polypeptide of any one of claims 19, 21 or 23 in a sample comprising the steps of:
a) contacting the sample with a compound which selectively binds to a phosphorylated polypeptide; and
b) determining whether the compound binds to the polypeptide in the sample to thereby detect the phosphorylated polypeptide in the sample.

30. The method of claim 29, wherein the compound which binds to the phosphorylated polypeptide is an antibody.

31. The method of claim 29, wherein the polypeptide is phosphorylated at one or more amino acids selected from the group consisting of: S1073, S1126, S1305, T1346, S1501, S1508, S1545 and S1552 of SEQ ID NO: 3.

32. A kit comprising a compound which selectively binds to the polypeptide of any one of claims 19, 21 or 23 when the polypeptide is phosphorylated, and instructions for use.

33. A method for detecting the nucleic acid molecule of any one of claims 2, 4, 6, 8 or 10 in a sample comprising the steps of:
a) contacting the sample with a nucleic acid probe or primer which selectively hybridizes to a complement of the nucleic acid molecule; and
b) determining whether the nucleic acid probe or primer binds to the complement of the nucleic acid molecule in the sample to thereby detect the presence of the nucleic acid molecule in the sample.

34. The method of claim 33, wherein the sample comprises mRNA molecules and is contacted with a nucleic acid probe.

35. A kit comprising a compound which selectively hybridizes to a complement of the nucleic acid molecule of any one of claims 2, 4, 6, 8 or 10 and instructions for use.

36. A method for identifying a compound which binds to a polypeptide of any one of claims 19, 21 or 23 comprising the steps of:
a) contacting the polypeptide with a test compound; and
b) determining whether the polypeptide binds to the test compound.

37. The method of claim 36, wherein the binding of the test compound to the polypeptide is detected by a method selected from the group consisting of:
a) detection of binding by direct detection of test compound/polypeptide binding;
b) detection of binding using a competition binding assay; and
c) detection of binding using an assay for separase activity.

38. The method of claim 37, wherein the separase activity is selected from the group consisting of: cleaving cohesin^{hSCC1}, cleaving separase, and modulating sister chromatid separation.

39. A method for modulating an activity of a polypeptide of any one of claims 19, 21 or 23 comprising contacting the polypeptide with an effective amount of a compound to modulate the activity of the polypeptide.

40. A method for identifying a compound that modulates an activity of a polypeptide of any one of claims 19, 21 or 23 comprising the steps of:
a) contacting the polypeptide with a test compound; and
b) determining a modulation of an activity of the polypeptide, thereby identifying a compound that modulates the activity.

41. The method of claim 40 wherein the activity is selected from the group consisting of: separase cleavage, cohesin^{SCC1} cleavage, and modulation of sister chromatid separation.

42. The method of claim 40 wherein separase phosphorylation is modulated.

43. A method for identifying a compound that modulates sister chromatid separation comprising the steps of:
a) contacting a polypeptide of any one of claims 19, 21 or 23 with the compound; and
b) determining a modulation of phosphorylation of the polypeptide, thereby identifying a compound that modulates sister chromatid separation.

44. The method of claim 43, wherein the phosphorylation occurs at one or more amino acid selected from the group consisting of: S1073, S1126, S1305, T1346, S1501, S1508, S1545, and S1552 of SEQ ID NO: 3

45. The method of claim 43, wherein the phosphorylation occurs at S1126 and/or T1346.

46. A method of modulating sister chromatid separation in a subject comprising administering to the subject a therapeutically effective amount of a compound identified in claim 40.

47. The method of claim 46, wherein the subject is a human.

48. The method of claim 46, wherein the compound is an antibody.

49. The method of claim 48, wherein the antibody is a phospho-specific antibody.

50. The method of claim 46, wherein the compound is an antisense molecule.

51. The method of claim 46, wherein the compound is a peptide.

52. The method of claim 46, wherein the compound is a small molecule.

53. The method of claim 46, wherein the compound inhibits sister chromatid separation.

54. The method of claim 46, wherein the compound enhances sister chromatid separation.

55. A method of treating a disorder in a subject comprising administering to a subject a therapeutically effective amount of a compound identified in claim 43.

56. The method of claim 55, wherein the disorder is cancer, Down's syndrome and/or spontaneous fetal abortion.

57. The method of claim 55, wherein the subject is a human.

58. The method of claim 55, wherein the compound is an antibody.

59. The method of claim 58, wherein the antibody is a phospho-specific antibody.

60. The method of claim 55, wherein the compound is an antisense molecule.

61. The method of claim 55, wherein the compound is a peptide.

62. The method of claim 55, wherein the compound is a small molecule.

63. The method of claim 55, wherein the compound inhibits sister chromatid separation.

64. The method of claim 55, wherein the compound enhances sister chromatid separation.

65. A method of modulating sister chromatid separation comprising contacting a polypeptide of any one of claims 19, 21 or 23 with an effective amount of a compound to modulate sister chromatid separation.

66. A method of modulating sister chromatid separation in a cell comprising contacting a cell expressing a polypeptide of any one of claims 19, 21 or 23 with an effective amount of a compound to modulate sister chromatid separation in the cell.

67. A method of modulating sister chromatid separation in a subject comprising administering to the subject a therapeutically effective amount of a nucleic acid of any one of claims 2, 4, 6, 8 or 10.

68. The method of claim 67, wherein the subject is a human.

69. The method of claim 67, wherein a polypeptide encoded by the nucleic acid has a mutation in one or more phosphorylation sites such that the site cannot be phosphorylated.

70. The method of claim 69, wherein the mutation occurs at an amino acid residue selected from the group consisting of: S1073, S1126, S1305, T1346, S1501, S1508, S1545, and S1552 of SEQ ID NO: 3

71. The method of claim 69, wherein the mutation occurs at S1126 and/or T1346.

72. A method of modulating sister chromatid separation in a subject comprising administering to the subject a therapeutically effective amount of a polypeptide of any one of claims 19, 21 or 23.

73. The method of claim 72, wherein the subject is a human.

74. The method of claim 72, wherein the polypeptide has a mutation in one or more phosphorylation sites such that the site cannot be phosphorylated.

75. The method of claim 74, wherein the mutation occurs at an amino acid residue selected from the group consisting of: S1073, S1126, S1305, T1346, S1501, S1508, S1545, and S1552 of SEQ ID NO: 3

76. The method of claim 74, wherein the mutation occurs at S1126 and/or T1346.

77. A method for identifying a compound that modulates an activity of a polypeptide of any one of claims 19, 21 or 23, wherein the polypeptide is expressed in a cell, comprising the steps of:
a) contacting a cell expressing the polypeptide with a test compound; and
b) determining a modulation of an activity of the polypeptide, thereby identifying a compound that modulates the activity.

78. The method of claim 77 wherein the activity is selected from the group consisting of: separase cleavage, cohesin^{SCC1} cleavage, and modulation of sister chromatid separation.
